(19) United States
(12) Patent Application Publication
(10) Pub. No.: US 2026/0315765 A1
(43) Pub. Date: Oct. 15, 2026

(54) METHOD AND DEVICE FOR SPRAYING COMPOSITION AND REFRIGERANT AFTER MICRO-HOLE FORMATION

(71) Applicant: RECENSMEDICAL, INC., Hwaseong-si (KR)

(72) Inventors: Gun-Ho KIM, Hwaseong-si (KR); Kyongkwan RO, Hwaseong-si (KR); Daehyun KIM, Hwaseong-si (KR)

(21) Appl. No.: 19/363,441

(22) Filed: Oct. 20, 2025

Related U.S. Application Data
(63) Continuation of application No. PCT/KR2024/005643, filed on Apr. 25, 2024.

(30) Foreign Application Priority Data
Apr. 25, 2023 (KR) 10-2023-0053940

(51) Int. Cl. A61M 37/00; A61M 11/00

(52) U.S. Cl. CPC A61M 37/0015; A61M 11/001; A61M 11/007; A61M 2037/0007; A61M 2037/0046; A61M 2202/0007; A61M 2202/04; A61M 2205/3331; A61M 2205/3368; A61M 2210/04

(57) ABSTRACT

Proposed is a method for spraying a composition and a coolant after micro-hole formation and a device therefor. In a method for mixing and spraying after needling, micro-scale holes are formed on the surface of the skin so that a composition effectively permeates the skin, and then the composition and a coolant are sprayed together. Also proposed is a needling device, a coolant spraying device, and a composition supply device, for performing the method for mixing and spraying after needling.

### CROSS-REFERENCE TO RELATED APPLICATIONS

This application is a continuation application, and claims the benefit under 35 U.S.C. § 120 and § 365 of PCT Application No. PCT/KR 2024/005643 filed on Apr. 25, 2024, which claims priority to Korean Patent Application No. 10-2023-0053940 filed on Apr. 25, 2023, the contents of each of which are hereby incorporated by reference in their entirety.

### BACKGROUND

### Technical Field

The present disclosure relates to a method of spraying a composition and a coolant after forming micro-holes and to a device configured therefor. More particularly, the present disclosure relates to a post-needling mixing spray method, in which a composition and a coolant are sprayed together after forming micro-scale holes in the skin surface to enable effective penetration of the composition into the skin, and to a device configured therefor.

### Description of Related Technology

In delivering a composition to the skin, the degree to which the composition penetrates into the skin has a significant impact on the degree of effect produced by the composition. Accordingly, various studies have been conducted to improve the penetration ability of compositions into the skin.

### SUMMARY

One aspect is to enable a composition to reach a depth beneath the epidermis of the skin.

Another aspect is to enable a composition to reach a depth beneath the stratum corneum of the skin.

Another aspect is to enable a composition to reach the dermis of the skin.

Another aspect is to form holes having a predetermined diameter and a predetermined depth in the skin surface before spraying a composition onto the skin surface.

Another aspect is to form holes in the skin surface, followed by spraying a coolant and a composition together.

Another aspect is to form holes in the skin surface, followed by freezing and spraying a composition.

The aspects to be solved are not limited to the foregoing, and other aspects not mentioned herein can be clearly understood by those skilled in the art to which the present disclosure pertains, from this specification and the accompanying drawings.

Another aspect a method for penetrating a composition into a depth beneath an epidermis of a skin, the method comprising: contacting a plurality of microneedles to a target region, wherein each of the plurality of microneedles has a pre-configured needle width and a needle length 0.1 mm or longer and 3.0 mm or shorter, wherein the contact between the plurality of microneedles and the target region forms a plurality of holes in the target region; and spraying, to the target region in which the plurality of holes are formed, a composition including an active ingredient in a liquid or emulsion type with a coolant, thereby lowering a temperature of the target region to below a body temperature while simultaneously impacting the composition to the target region.

Another aspect is a needling and mixing spray system, the needling and mixing spray system comprising: a coolant container configured to accommodate a coolant; a coolant receiving unit configured to receive the coolant from the coolant container; a valve fluidically connected to the coolant receiving unit, wherein the coolant output from the coolant receiving unit flows into the valve; a nozzle fluidically connected to the valve and having an orifice of a pre-configured diameter, wherein the coolant output from the valve flows into the nozzle and a coolant spray stream is formed as a coolant passes through the nozzle; a composition container configured to accommodate a composition; a composition guide fluidically connected to the composition container and providing a pathway through which a composition moves, wherein an end of the composition guide disposed adjacent to the nozzle, a composition is introduced into a coolant spray stream through the composition guide when the coolant spray stream is formed by the nozzle; and a needling structure including a body part and a plurality of needles protruding from the body part, wherein the needling structure is disposed at a predetermined distance from the orifice of the nozzle and is disposed such that a central axis of the nozzle does not pass through the needling structure.

The effects of the present disclosure are not limited to those mentioned above, and other effects not mentioned will be clearly understood by those skilled in the art from the following description and the accompanying drawings.

According to an embodiment, a composition can reach a depth beneath the epidermis of the skin.

According to an embodiment, a composition can reach a depth beneath the stratum corneum of the skin.

According to an embodiment, a composition can penetrate the skin through holes formed in the skin surface.

According to an embodiment, pain can be relieved during a process of forming holes in the skin surface.

According to an embodiment, the amount of composition that penetrates the skin can be increased with respect to the amount of the composition provided to the skin surface.

The effects of the present disclosure are not limited to the foregoing, and other effects not mentioned herein can be clearly understood by those skilled in the art to which the present disclosure pertains, from this specification and the accompanying drawings.

### BRIEF DESCRIPTION OF THE DRAWINGS

FIG. 1 is a schematic diagram illustrating a mixing spray system according to an embodiment.

FIG. 2 is a diagram illustrating a needling device, according to an embodiment, and its use.

FIG. 3 is a diagram illustrating a post-needling mixing spray method performed according to an embodiment.

FIG. 4 is a flowchart illustrating a post-needling mixing spray method according to an embodiment.

FIG. 5 is a diagram illustrating a post-needling mixing spray method performed according to an embodiment, with a target region being divided.

FIGS. 6 to 8 are diagrams showing the results of a first experiment to confirm the penetration effects of an application method and a mixing spray method in providing a composition to the skin.

FIGS. 9 to 11 are diagrams showing the results of a second experiment to confirm the penetration effects of an application method, a mixing spray method, a needling method, and a post-needling mixing spray method in providing a composition to the skin.

FIGS. 12 to 14 are diagrams showing the results of a third experiment to confirm the penetration effects of an application method, a mixing spray method, a needling method, and a post-needling mixing spray method in providing a composition to the skin.

FIG. 15 is a diagram illustrating how a composition is frozen and sprayed according to an embodiment.

FIG. 16 is a diagram illustrating how the freeze ratio of a composition is determined using an observing region according to an embodiment.

FIGS. 17 and 18 are images of a scene in which a liquid composition and a solid composition are sprayed according to an embodiment.

FIG. 19 is a diagram showing an image processing process for counting solid composition particles according to an embodiment.

FIG. 20 is a diagram illustrating how the freeze ratio is calculated using an observing line according to an embodiment.

FIG. 21 is a diagram illustrating a mixing spray system according to an embodiment.

FIG. 22 is a diagram illustrating components of a coolant spraying device according to an embodiment.

FIG. 23 is a diagram illustrating a composition supply device according to an embodiment.

FIG. 24 is a diagram illustrating a composition supply device according to another embodiment.

FIG. 25 is a flowchart illustrating a freeze spray method according to an embodiment.

FIGS. 26 and 27 are diagrams showing the results of a first experiment to confirm the penetration effect in the presence or absence of freezing and the penetration effect depending on the freeze ratio.

FIG. 28 is a diagram showing the results of a second experiment to confirm the penetration effect depending on the freeze ratio.

FIG. 29 is a diagram showing the penetration effects of Test Groups 1 and 5 in a second experiment.

FIG. 30 is a diagram showing the relationship between particle size and spray speed according to an embodiment.

FIG. 31 is a diagram illustrating a needling and mixing spray system according to an embodiment.

FIG. 32 is a diagram illustrating a form in which a needling and mixing spray system is implemented according to an embodiment, and its use.

### DETAILED DESCRIPTION

Methods for delivering compositions to the skin include applying a composition to the skin, injecting a composition into the skin, or spraying a composition onto the skin.

Of these, it is known that the applying of the composition to the skin makes it difficult for the composition to pass through the lipid layer of the skin depending on its molecular weight and biochemical properties, and as a result, only a very small amount is absorbed into the skin.

In addition, the injecting of the composition into the skin allows the composition to penetrate deep into the skin, but inevitably damages the skin. This may cause not only pain during procedure but also problems such as bruising or swelling due to damage to the skin. Furthermore, the injecting of the composition into the skin requires operator's skill, so the penetration depth and amount may vary depending on the operator. This is disadvantageous in that the effect of the composition may be inconsistent.

Thus, there is a need for a composition delivery method that ensures the penetration ability of the composition into the skin and induces a consistent penetration effect while minimizing damage to the skin.

According to an embodiment, a method for penetrating a composition into a depth beneath an epidermis of a skin is provided. the method comprising: contacting a plurality of microneedles to a target region, wherein each of the plurality of microneedles has a pre-configured needle width and a needle length 0.1 mm or longer and 3.0 mm or shorter, wherein the contact between the plurality of microneedles and the target region forms a plurality of holes in the target region; and spraying, to the target region in which the plurality of holes are formed, a composition including an active ingredient in a liquid or emulsion type with a coolant, thereby lowering a temperature of the target region to below a body temperature while simultaneously impacting the composition to the target region.

The lowering the temperature of the target region to below the body temperature while simultaneously impacting the composition to the target region is performed within a pre-configured time after the contacting the plurality of microneedles to the target region.

The pre-configured time has a value between 10 seconds and 20 seconds.

The contacting the plurality of microneedles to the target region comprising: dividing the target region into at least a first region and a second region; sequentially contacting the plurality of microneedles to the first region and the second region, wherein the composition and the coolant are not sprayed to the first region during the plurality of microneedles is contacted to the second region.

A needling device including a roller having the plurality of microneedles protruding from an outer surface is used in the contacting the plurality of microneedles to the target region, and wherein the plurality of microneedles contacts the target region while the roller is rolled on the target region thereby forming the plurality of holes.

The needle length is 1.5 mm.

The needle length is 0.5 mm.

A mixing spray system is used in the spraying the composition with the coolant to the target region, and wherein the mixing spray system comprises a coolant spray device including a nozzle for spraying the coolant and a composition supply device configured to introduce a composition into a coolant spray stream formed by the nozzle.

A portion of the composition sprayed to the target region is frozen by a coolant and reaches to the target region in a solid state.

A freezing rate of the composition reached to the target region is 5 % or more.

According to an embodiment, a needling and mixing spray system is provided. The needling and mixing spray system comprising: a coolant container configured to accommodate a coolant; a coolant receiving unit configured to receive the coolant from the coolant container; a valve fluidically connected to the coolant receiving unit, wherein the coolant output from the coolant receiving unit flows into the valve; a nozzle fluidically connected to the valve and having an orifice of a pre-configured diameter, wherein the coolant output from the valve flows into the nozzle and a coolant spray stream is formed as a coolant passes through the nozzle; a composition container configured to accommodate a composition; a composition guide fluidically connected to the composition container and providing a pathway through which a composition moves, wherein an end of the composition guide disposed adjacent to the nozzle, a composition is introduced into a coolant spray stream through the composition guide when the coolant spray stream is formed by the nozzle; and a needling structure including a body part and a plurality of needles protruding from the body part, wherein the needling structure is disposed at a predetermined distance from the orifice of the nozzle and is disposed such that a central axis of the nozzle does not pass through the needling structure.

A region corresponding to the nozzle and a region corresponding to the needling structure do not overlap when viewed from a front of the nozzle.

The needling structure is disposed at a first distance from the orifice of the nozzle in a direction parallel to the central axis of the nozzle, and at a second distance from the central axis of the nozzle.

The first distance has a value between 3 mm and 100 mm.

The needling and mixing spray system further comprises: an additional needling structure, wherein the additional needling structure is disposed such that the central axis of the nozzle does not pass through the additional needling structure, and wherein the central axis of the nozzle is located between the needling structure and the additional needling structure.

The above-described objectives, features, and advantages of the present disclosure will be more clearly understood from the following detailed description when taken in conjunction with the accompanying drawings. The present disclosure may, however, be embodied in many different forms and should not be limited to the exemplary embodiments. Hereinafter, reference will now be made in more detail to embodiments, specific examples of which are illustrated in the accompanying drawings.

In the drawings, the thicknesses of layers and regions are exaggerated for clarity. Further, when an element or layer is referred to as being “on” or “above” another element or layer, it can be directly above the other element or layer or intervening elements or layers may be present therebetween. In principle, like reference numerals designate like elements throughout the specification. In addition, like reference numerals are used to designate elements which have the same function within the same idea illustrated in the drawings of each embodiment, and overlapping descriptions thereof will be omitted.

Numerals (e.g., first, second, etc.) used in the description herein are merely identifiers for distinguishing one element from another element.

In addition, the element suffixes “module” and “part” used in the following embodiments are given or mixed together only considering the ease of creating the specification, and have no meanings or roles that are distinguished from each other by themselves.

As used herein, the singular forms “a”, “an”, and “the” are intended to include the plural forms as well, unless the context clearly indicates otherwise.

In the following embodiments, the terms “comprise”, “include”, “have”, etc. when used in this specification, specify the presence of stated features or elements, but do not preclude the presence or addition of one or more other features or elements.

In the drawings, the sizes of elements may be exaggerated or reduced for convenience of explanation. For example, the sizes and thicknesses of elements in the drawings are arbitrarily illustrated for convenience of explanation, and embodiments of the present disclosure are not limited thereto.

When some example embodiments may be embodied otherwise, specific process steps described herein may be performed otherwise. That is, for example, two process steps described in a sequential order may be performed around the same time, or in reverse order.

In the following embodiments, when films, regions, elements, etc. are referred to as being connected to each other, they can be directly connected or be indirectly connected to each other with one or more intervening films, regions, elements, etc. interposed therebetween.

For example, when films, regions, elements, etc. are referred to as being electrically connected to each other, they can be electrically directly connected or be electrically indirectly connected to each other with one or more intervening films, regions, elements, etc. interposed therebetween.

In the following embodiments, when films, regions, elements, etc. are referred to as being fluidly connected to each other, it may be understood that they may form at least a part of a flow path through which each fluid flows.

For example, that an element A is fluidly connected to an element B may mean that a fluid passing through a flow path formed by the element A may reach a flow path formed by the element B or vice versa. Specifically, when the element A and the element B are coupled and the flow path formed by the element A and the flow path formed by the element B are directly connected to each other, the element A and the element B can be considered to be fluidly connected to each other. Otherwise, when the element A and element B are connected to each other through an element C, such as a conduit, so that the flow path formed by the element A and the flow path formed by the element B are indirectly connected to each other through a flow path formed by the element C, the element A and the element B can be considered to be fluidly connected to each other. Here, the element C can be understood as fluidly connecting the elements A and B. Furthermore, the element A and the element B may be fluidly connected to each other through a plurality of elements.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The present disclosure relates to a method of spraying a composition and a coolant after forming micro-holes and to a device configured therefor. More particularly, the present disclosure relates to a post-needling mixing spray method, in which a composition and a coolant are sprayed together after forming micro-scale holes in a target region to enable effective penetration of the composition into the skin, and to a device configured therefor.

The composition may include an active substance that induces or produces a medical effect. Alternatively, the composition may include an active substance that induces or produces a cosmetic effect.

Furthermore, the composition in the present disclosure is particularly characterized by transdermal delivery, and the composition may refer to a substance that produces a cosmetic or medical effect during transdermal delivery.

The composition may include, for example, mineral, nucleic acid, amino acid, coenzyme, vitamin, niacinamide, oil-soluble licorice extract, arbutin, hyaluronic acid, potassium hyaluronate, hydrolyzed hyaluronic acid, hydrolyzed sodium hyaluronate, hydroxypropyltrimonium hyaluronate, sodium acetylated hyaluronate, sodium hyaluronate crosspolymer, sodium hyaluronate, retinol, retinyl palmitate, adenosine, peptide, coenzyme Q10, adult stem cell, antioxidant, poly-d-lactic acid (PDLA), polynucleotide, polydeoxyribonucleotide (PDRN), poly-d, l-lactic acid (PDLLA), lidocaine, botulinum toxin, exosome, proparacaine, tetracaine, human growth hormone, growth factor, cell therapy products, or a combination thereof.

Furthermore, the composition may further include: base ingredients such as purified water, glycerin, butylene glycol, propanediol, and silicone oil; ingredients that create formulations such as emulsifier, surfactant, and viscosity modifier; and preservative ingredients such as paraben, phenoxyethanol, benzoic acid, triclosan, benzyl alcohol, methylisothiazolinone, and 1,2-hexanediol.

In the present disclosure, the use of a transfer medium as a method of spraying the composition. For example, the composition may be carried by a coolant sprayed at a relatively high velocity and sprayed onto the target region. In this case, the temperature of the composition may be lowered by the coolant sprayed at a relatively low temperature.

Here, the coolant may be liquefied carbon dioxide (CO₂), carbon dioxide, liquefied nitrogen, liquefied oxygen, nitrogen dioxide (NO₂), nitrogen monoxide (NO), nitrous oxide (N₂O), hydrofluorocarbon (HFC) series materials, methane (CH₄), perfluorocarbon (PFC), sulfur hexafluoride (SF₆), or a combination thereof, and materials that can apply cooling energy to the target region, such as coolant and cooling gas, may be used. Meanwhile, in addition to the coolant, compressed air, etc. may be used.

Hereinafter, for convenience of explanation, it will be described that the composition is carried by the coolant and liquefied carbon dioxide is used as the coolant, but the technical idea of the present disclosure is not limited thereto.

As used herein, needling refers to forming a plurality of holes in a specific region. For example, needling may refer to forming a plurality of holes in a target region by contacting the target region with a plurality of needles.

In the present disclosure, the target region refers to a region where the composition needs to be sprayed. Specifically, the target region may refer to the skin surface of a part of the human body. Alternatively, the target region may refer to the skin surface of a part of the body of an animal other than a human. The target region may be determined depending on the body part to which the composition is to be provided.

The post-needling mixing spray method described herein has various uses as follows.

The post-needling mixing spray method may be used as a treatment method or medical procedure. The composition referring to a drug or a pharmaceutical, in this case, may be understood as containing a substance used for the diagnosis, cure, alleviation, treatment, or prevention of a disease or disorder, skin regeneration, skin lifting, and treatment of skin problems such as acne and inflammation.

Physicians or others (healthcare providers with medical expertise defined under medical laws in each country) may use the post-needling mixing spray method to perform needling on a target region of a subject and then spray, together with a coolant, a composition having a therapeutic effect on a specific disease or disorder.

The post-needling mixing spray method may be used as a cosmetic method, as a cosmetic procedure, or for skin care. The composition referring to a cosmetic product, in this case, may be understood as containing a substance capable of inducing cosmetic effects such as skin brightening, soothing, nourishment, moisturizing, wrinkle reduction, or elasticity improvement when applied to the skin. Furthermore, the composition may be composed of substances that have almost no or little effect on humans.

Estheticians or skin care professionals may use the post-needling mixing spray method to perform needling on a target region of a subject and then spray, together with a coolant, a composition having a cosmetic effect.

In the meantime, individuals may also use the post-needling mixing spray method to induce cosmetic effects on their own skin. In this case, a needling device for individual use may have a needle length not exceeding a limited length (for example, 0.25 mm), as will be described below.

The post-needling mixing spray method may be used as a treatment method or cosmetic method for animals other than humans. In this case, The composition referring to a drug, a pharmaceutical, or a cosmetic product may be understood as containing an active ingredient having a therapeutic or cosmetic effect.

### Regarding Composition Penetration

The main purpose of the freeze spray method to be described in the present disclosure is to enable a composition to penetrate into the skin.

### Meaning and Significance of Composition Penetration

Penetration of the composition means that the composition reaches a specific depth from the skin surface. More specifically, penetration of the composition means that the composition reaches a depth below the epidermis of the skin.

The epidermis is composed of stratum corneum, transparent stratum, stratum granulosum, stratum spinosum, and stratum basale. In particular, the stratum corneum functions as a skin barrier and is the main factor that hinders absorption of the composition.

Therefore, penetration of the composition means that the composition reaches at least below the stratum corneum. Since the thickness of the stratum corneum is about 10 μm to about 20 μm, penetration of the composition means that the composition reaches a depth of about equal to or greater than 0.01 mm from the skin surface.

For example, when a target to which the composition is to be provided is the face of a Korean adult, the epidermis is distributed to a depth of about 0.1 mm from the skin surface, and the dermis is distributed to a depth of about 0.1 mm to about 2 mm from the skin surface.

Penetration of the composition into the face means that from the skin surface, the composition reaches a depth of about 0.01 mm to about 2 mm, a depth of about 0.02 mm to about 2 mm, a depth of about 0.1 mm to about 2 mm, a depth of about 0.2 mm to about 2 mm, a depth of about 0.3 mm to about 2 mm, a depth of about 0.4 mm to about 2 mm, a depth of about 0.5 mm to about 2 mm, a depth of about 0.6 mm to about 2 mm, a depth of about 0.7 mm to about 2 mm, a depth of about 0.8 mm to about 2 mm, a depth of about 0.9 mm to about 2 mm, a depth of about 1 mm to about 2 mm, a depth of about 1.1 mm to about 2 mm, a depth of about 1.2 mm to about 2 mm, a depth of about 1.3 mm to about 2 mm, a depth of about 1.4 mm to about 2 mm, a depth of about 1.5 mm to about 2 mm, a depth of about 1.6 mm to about 2 mm, a depth of about 1.7 mm to about 2 mm, a depth of about 1.8 mm to about 2 mm, or a depth of about 1.9 mm to about 2 mm. Since the thickness of the epidermis and dermis may vary depending on the area in the face, a reference depth defining penetration of the composition is not limited to the above-described values.

Meanwhile, the target to which the composition is sprayed is not limited to the face, and other body parts other than the face, such as the scalp, neck, arms, legs, hands, and feet, may be the target. Here, penetration of the composition also means that the composition reaches below the stratum corneum or epidermis of each body part.

In order for the composition to achieve its purpose (e.g., inducing a therapeutic effect or cosmetic effect), it is important that it is absorbed into the skin. In order for the composition to be absorbed into the skin, the composition needs to reach and spread beyond the stratum corneum of the skin to the stratum basale or dermis. In other words, even when the composition includes an active substance with high effectiveness, the effect will inevitably be minimal when it stays in the stratum corneum of the skin.

There are three major methods of enabling the composition to penetrate into the dermis through the epidermis of the skin. They include a transcellular route, which passes directly through the stratum corneum of the epidermis, an intercellular route, which passes through the non-polar lipid layer between corneocytes, and a trans-appendageal route, which passes through appendages such as pores.

However, since the appendages occupy 0.1% of the skin surface area, the amount of penetration through the appendages is limited. Therefore, penetration through the transcellular or intercellular route is more important than the trans-appendageal route.

### Conventional Composition Penetration Methods and Problems

Conventional methods for penetrating compositions into the skin include an application method of applying a composition, an injection method using a syringe, a needling method of forming holes in the skin with a microneedle after applying a composition, and a method of spraying a composition in the form of microparticles.

The application method of applying the composition to the skin surface is to enable the composition to penetrate through the transcellular or intercellular route. However, even when the applied composition passes through the stratum corneum through the transcellular route, it is blocked by the stratum granulosum of the epidermis and thus difficult to pass through the stratum spinosum or stratum basale, and as a result, it is difficult for the composition to reach the dermis. Even when the composition passes through the stratum corneum through the intercellular route, the amount of passed-through composition is very small, so the amount of the composition that reaches the dermis is not sufficient.

The injection method using the syringe is to invasively deliver the composition by inserting the syringe into the dermis or subcutaneous tissue. The injection method using the syringe is accompanied by relatively strong pain compared to other penetration methods, and is problematic in that the composition is concentratedly introduced into one point of the skin and does not spread evenly over the skin. In addition, in the case of the syringe, the position where the composition is introduced into the skin varies depending on the degree to which the syringe is inserted into the skin. Since the total thickness of the epidermis and dermis is about 2 mm, accurately inserting the syringe into the dermis is dependent on the operator's skill level, and therefore a consistent treatment effect may not be guaranteed.

The needling method involves applying the composition to the skin, forming holes in the skin using a needling device equipped with a plurality of microneedles having a micro-level length, and enabling the composition to penetrate through the holes.

In the case of the needling method, the microneedles are inserted into the skin with the composition applied thereto, or the composition penetrates into the skin through the holes formed by the microneedles. Due to the thickness of the microneedles, the holes formed in the skin are also thin, and therefore, the amount of composition that is introduced through the holes is limited.

In addition, the needling method may be accompanied by pain depending on the needle size of the needling device, and the skin is damaged as the needling device forms the holes in the skin, so additional care to restore skin damage is required after penetration of the composition. In addition, sterilization before use is essential to prevent infection caused by the needles of the needling device.

The airbrush method uses compressed air as a transfer medium and uses a fine nozzle 1500 to make the composition into relatively small microparticles and spray them on the skin. The airbrush method is a non-invasive method of providing the composition, is painless, and is easy to use. However, in the case of the airbrush method, the composition collides with the skin surface in a liquid state, making it difficult to provide an impact strong enough to physically pierce the epidermis. Consequently, in the airbrush method, the composition passes through the stratum corneum through the transcellular or intercellular route, but like the application method, it is difficult for the composition to pass through the stratum spinosum or stratum basale of the epidermis.

### Composition Delivery Method Using Coolant

As a method of penetrating the composition into the skin, the present disclosure will describe a method using a mixing spray system 100.

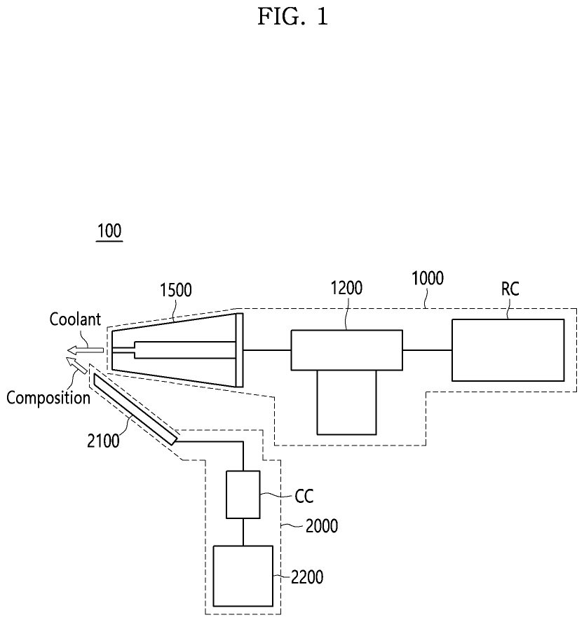

FIG. 1 is a schematic view illustrating a mixing spray system 100 according to an embodiment.

The mixing spray system 100 adopts a method of spraying a composition using a coolant. Referring to FIG. 1, the mixing spray system 100 may include a coolant spray device 1000 and a composition supply device 2000.

The coolant spray device 1000 refers to a device that sprays the coolant. The coolant spray device 1000 includes at least a coolant container RC in which the coolant is stored, a flow regulating unit 1200 that controls movement of the coolant so that the coolant is sprayed or not sprayed from the coolant spray device 1000, and a nozzle 1500 through which the coolant is sprayed. The coolant spray device 1000 may further include other components required for operation in addition to those described above. Additional components of the coolant spray device 1000 will be described later.

The composition supply device 2000 refers to a device that provides the composition. The composition supply device 2000 may include a composition container CC that stores the composition and a composition guide 2100 that discharges the composition. The composition guide 2100 may include an input end through which the composition stored in the composition container CC is introduced, and an output end through which the composition is discharged. The composition supply device 2000 may further include other components required for operation in addition to those described above. Additional components of the composition supply device 2000 will be described later.

The composition supply device 2000 may be connected to the coolant spray device 1000 and provide the composition to the coolant sprayed from the coolant spray device 1000. Specifically, a coolant spray stream may be formed by the nozzle 1500 of the coolant spray device 1000, and the composition guide 2100 of the composition supply device 2000 may be disposed adjacent to the nozzle 1500.

Here, the coolant spray stream refers to a coolant flow including coolant particles sprayed from the nozzle 1500. The coolant spray stream formed by the nozzle 1500 may form a negative pressure at the output end of the composition guide 2100, and the negative pressure may enable the composition to be moved along the composition guide 2100 and introduced into the coolant spray stream. Alternatively, the composition supply device 2000 may include an actuator 2200 fluidly connected to the composition container CC. The composition may be supplied to the composition guide 2100 at a predetermined flow rate or a flow rate within a predetermined range by the actuator 2200 and introduced into the coolant spray stream.

The composition introduced into the coolant spray stream may collide with coolant particles in the coolant spray stream and be sprayed together. The composition in the coolant spray stream may be broken into small microparticles by the high velocity of the coolant spray stream and cooled by heat exchange with the coolant spray stream having a relatively low temperature. As such, the composition and the coolant may be mixed and sprayed in the mixing spray system 100.

Meanwhile, the internal pressure of the coolant container RC in which the coolant is stored may be about 10 bar to about 1000 bar at room temperature. Alternatively, the internal pressure of the coolant container RC may be about 30 bar to about 200 bar at room temperature. Alternatively, the internal pressure of the coolant container RC may be about 50 bar at room temperature. The internal pressure of the coolant container RC may be related to the velocity at which the coolant expands when it is discharged from the nozzle 1500 of the coolant spray device 1000. In other words, as the internal pressure of the coolant container RC increases, the velocity of coolant particles in the coolant spray stream may increase. Considering that the pressure of compressed air used in the above-described airbrush method is about 1 bar to about 5 bar, using a high-pressure coolant as a transfer medium can significantly increase the spray speed of the composition, thereby improving the penetration effect of the composition.

However, even after reviewing various existing literature, it was not known that the composition could effectively penetrate into the skin when the state of the composition reaching the skin surface is controlled using the mixing spray system 100. In particular, it was not known that the composition could effectively penetrate into the skin when it reached the skin surface in a “frozen” state.

There has been a history of experiments suggesting that spraying carbon dioxide and the composition together has an effect in reducing cellulite. However, this is interpreted as a cooling effect due to carbon dioxide, and does not reveal that a larger amount of composition “reached” fat cells, so it cannot provide an indicator suggesting that a relatively large amount of composition penetrated into the skin.

In the course of conducting research on the mixing spray system 100, the applicant of this application has discovered that the composition in the mixing spray system 100 is absorbed according to the principle that “the composition is sprayed at a high speed and penetrates the epidermis while forming holes therein”.

Hence, the applicant has hypothesized that the penetration ability of the composition into the skin can increase when the needling method is performed together with the mixing spray method using the mixing spray system 100. This is because not only does the needling method involve forming a channel through which the composition moves to the epidermis from the skin surface, between the epidermis and the dermis, or to the dermis, but also the mixing spraying involves forming holes in the skin.

In other words, the applicant has drawn a conclusion that, because both methods involve forming a channel physically penetrating the epidermis of the skin, the effect can be significantly improved when these methods are performed together.

However, there is a possibility that, when the composition is sprayed together with the coolant during the mixing spraying, the coolant may interfere with the penetration of the composition compared to when the composition is sprayed alone, as well as a possibility that lowering the temperature of the skin surface by the coolant may affect the penetration of the composition. For this reason, the applicant has determined that the possibility of the penetration ability being rather reduced when the needling method and the mixing spray method are performed together cannot be ruled out. Accordingly, the applicant has designed and conducted experiments to determine the difference between performing the needling method together with the mixing spraying and separately performing the needling method and mixing spraying.

### Post-Needling Mixing Spraying

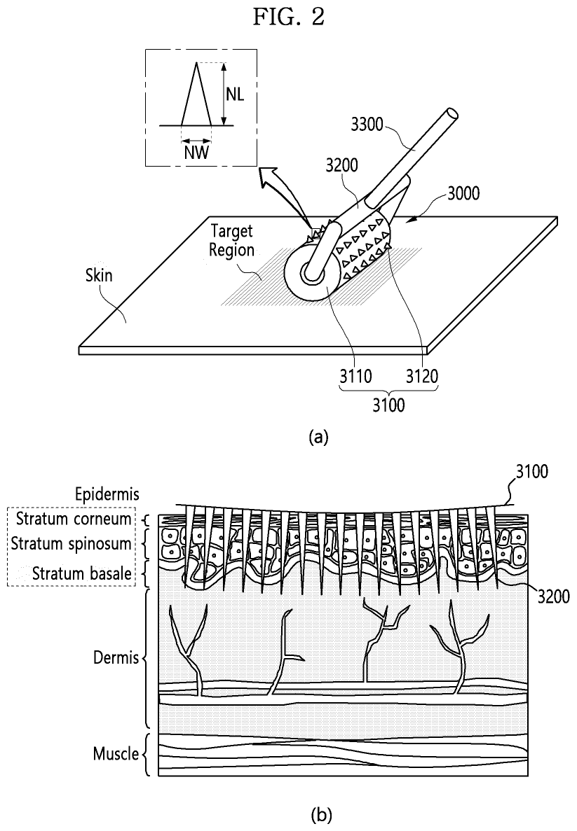

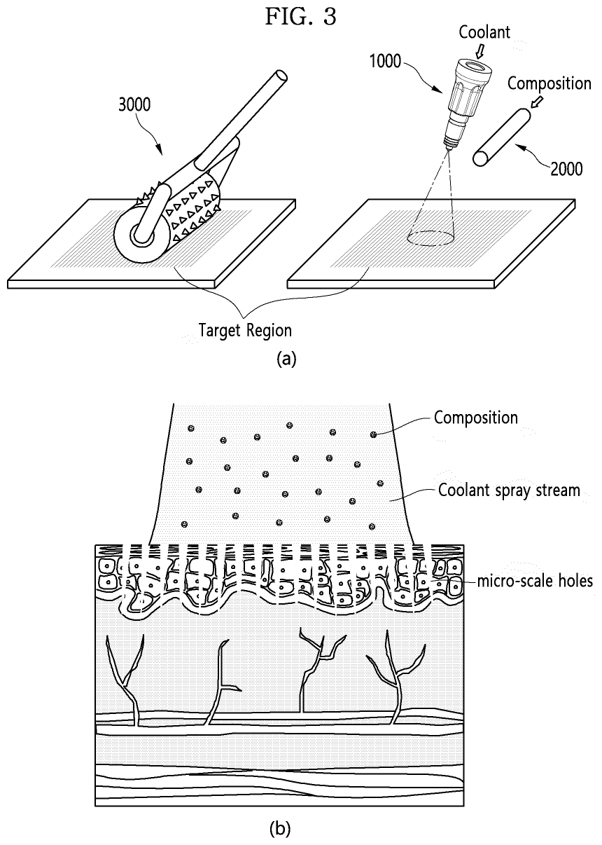

Hereinafter, a needling device is to be described with reference to FIG. 2. In addition, a post-needling mixing spray method is to be described with reference to FIGS. 3 to 5.

FIG. 2 is a diagram illustrating a needling device 3000, according to an embodiment, and its use. While (a) of FIG. 2 illustrates the use of the needling device 3000 on a target region, (b) of FIG. 2 illustrates a cross-section of the skin subject to needling by the needling device 3000.

### Needling Device and Method of Use

The needling device 3000 is a device configured to form holes in the target region. The needling device 3000 can be used in various ways. In one example, a practitioner may perform rolling on the target region with the needling device 3000. In another example, a practitioner may perform stamping on the target region with the needling device 3000. In a further example, with the needling device 3000 positioned on the target region, a practitioner may also press a trigger button so that the needling device 3000 automatically punches the target region, thereby forming holes.

Hereinafter, for convenience of explanation, although a case where the needling device 3000 is used in a rolling manner is to be described, the technical idea of the present disclosure is not limited thereto, and the needling device 3000 may also be used in a stamping manner or an automatic punching manner, as described above.

Referring to FIG. 2, the needling device 3000 may include a needling structure 3100, a connecting frame 3200, and a grip member 3300.

The needling structure 3100 forms holes in the target region when the needling device 3000 is used on the target region. The needling structure 3100 can be divided into a body part 3110 and a needle array 3120.

The body part 3110 may include a contact surface contacting with the target region when forming holes in the target region using the needling device 3000.

The body part 3110 may have a shape suitable for rolling. In one example, the body part 3110 may have a cylindrical shape enabling rotation around a central axis. In another example, the body part 3110 may have a polygonal column shape. Accordingly, the needling structure 3100 may be referred to as a roller.

The body part 3110 may have a body width and a body length. When the body part 3110 has a cylindrical or polygonal column shape, the body width refers to the diameter or width of the column, and the body length refers to the height of the column.

The needle array 3120 includes a plurality of needles, and each needle has a sharp shape to form a hole. The needle array 3120 may protrude from the body part 3110.

Each needle included in the needle array 3120 has a needle length NL and a needle width NW on the body part 3110. The needle length NL is, for example, in the range of about 0.1 mm to 3 mm. The needle width NW is, for example, in the range of about 10 μm to 300 μm. Accordingly, each hole formed in the target region using the needling device 3000 may have a width in the range of about 10 μm to about 300 um and a depth in the range of about 0.1 μm to about 3 mm.

In the meantime, the needle length NL can be classified depending on whether the device is intended for home use by an individual or for hospital use in performing therapeutic or cosmetic procedures. In one example, the needling device 3000 intended for home use may have a needle length NL of 0 mm or greater and about 0.25 mm or smaller, while the needling device 3000 intended for hospital use may have a needle length NL of 0.25 mm or greater and 3 mm or smaller.

The respective needles in the needle array 3120 may differ from each other in needle length NL or needle width NW. Depending on the needle length NL and needle width NW values of the needle array 3120, the needle array 3120 may be called a microneedle array, a nanoneedle array, or the like. Furthermore, the needling device 3000 may be called a microneedle device, a nanoneedle device, or the like.

The connecting frame 3200 refers to a component configured to connect the needling structure 3100 and the grip member 3300.

The grip member 3300 refers to a part gripped by a user when using the needling device 3000.

The needling device 3000 may be used in a rolling manner on the target region. In one example, as shown in (a) of FIG. 2, the user may position the needling device 3000 on the target region while gripping the grip member 3300 of the needling device 3000, and roll the needling structure 3100.

With the needling structure 3100 being rolled on the target region, the needle array 3120 may penetrate the target region, as shown in (b) of FIG. 2. In this case, depending on the needle length NL, holes may be formed in the skin, reaching the dermis.

The needling device 3000 may be implemented in such a manner that needling is automatically performed.

Specifically, the needling structure 3100 of the needling device 3000 may have the body part 3110 including the contact surface for contacting with the target region. A plurality of holes may be formed in the contact surface, and the plurality of needles may protrude through the plurality of holes in response to a trigger input. More specifically, when the needling device 3000 receives a trigger input while the body part 3110 of the needling device 3000 is in contact with the target region by the user, the plurality of needles may repeatedly protrude from and retract into the holes of the body part 3110 at a preset cycle, thereby performing needling on the target region. The needling device 3000 may further include a power module configured to move the plurality of needles and a control module configured to control the same.

### Post-Needling Mixing Spray Method

FIG. 3 is a diagram illustrating the post-needling mixing spray method performed according to an embodiment. In FIG. 3, only some components of a mixing spray system 100 are shown for convenience of explanation.

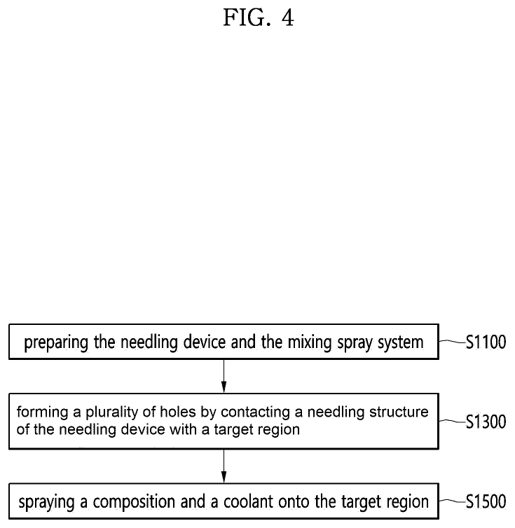

FIG. 4 is a flowchart illustrating the post-needling mixing spray method according to an embodiment.

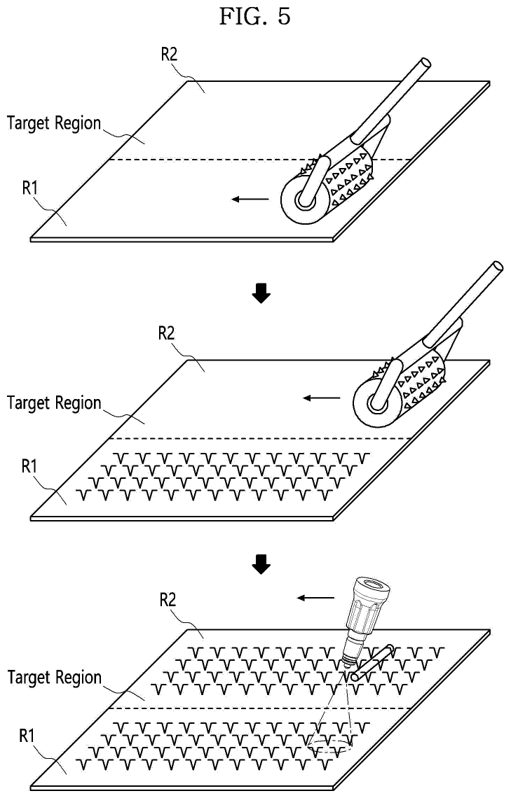

FIG. 5 is a diagram illustrating the post-needling mixing spray method performed according to an embodiment, with the target region being divided.

As shown in (a) of FIG. 3, the user may form a plurality of holes in the target region using the needling device 3000, and spray the composition and the coolant using the mixing spray system 100.

As shown in (b) of FIG. 3, the plurality of holes may be formed in the skin using the needling device 3000. By spraying the composition into the plurality of holes, the composition may reach a depth beneath the epidermis of the skin.

Referring to FIG. 4, the post-needling mixing spray method may include the following steps: preparing a needling device 3000 and a mixing spray system 100 (S1100); forming a plurality of holes by contacting a needling structure 3100 of the needling device 3000 with a target region (S1300); and spraying a composition and a coolant onto the target region (S1500).

Hereinafter, each step is to be described.

First, the user may prepare the needling device 3000 and the mixing spray system 100 (S1100).

The needling device 3000 is a device including a needle configured to form a hole, as described above, and may be of a roller type, a stamp type, or a punching (automatic needling) type for use.

The needle length NL of the needling structure 3100 may be in the range of about 0.1 mm to about 3 mm. Preferably, the needle length NL is 0.25 mm, 0.5 mm, or 1.5 mm. When the composition intended to penetrate the target region is not a pharmaceutical, the needling device 3000 may be selected from those having a needle length NL of 0.5 mm or smaller or 0.25 mm or smaller.

The needle width NW of the needling structure 3100 is, for example, in the range of about 10 μm to 300 μm.

The mixing spray system 100 refers to a device configured to spray the coolant and the composition together. The mixing spray system 100 may include the components described above. In one example, the mixing spray system 100 may at least include a coolant container RC, a flow regulating unit 1200, a nozzle 1500, a controller 1900, a composition container CC, and a composition guide 2100, wherein the coolant is sprayed from the nozzle 1500 when the flow regulating unit 1200 is opened, and the composition is introduced into and sprayed onto a coolant spray stream RSS formed at the nozzle 1500.

The user may form the plurality of holes by contacting the needling structure 3100 of the needling device 3000 with the target region (S1300). In one example, when using a roller-type needling device 3000, the user may form a plurality of holes in the skin by rolling the needling structure 3100 of the needling device 3000 while being in contact with the target region. In another example, when using a stamp-type needling device 3000, the user may contact the needling structure 3100 of the needling device 3000 with the target region while being pressed in the direction of contact, thereby forming a plurality of holes in the skin. In a further example, when using a punching-type needling device 3000, the user may operate the needling device 3000 while contacting the needling structure 3100 of the needling device 3000 with the target region, thereby forming a plurality of holes in the skin.

The plurality of holes formed in the target region may have a depth and a width corresponding to the needle length NL and the needle width NW of the needling device 3000, respectively.

The user may spray the composition and the coolant onto the target region (S1500). Referring to (a) of FIG. 3, the user may spray the coolant and the composition while directing the nozzle 1500 of a coolant spraying device 1000 toward the target region.

The mixing spraying of the composition and the coolant may be performed within a preset time after forming the plurality of holes in the target region. In this case, the preset time may be determined within about 10 seconds. The preset time may, for example, be 0.5 seconds, 1 second, 2 seconds, 3 seconds, 4 seconds, 5 seconds, 6 seconds, 7 seconds, 8 seconds, 9 seconds, or 10 seconds.

In the target region, a needling region in contact with the needling structure 3100 of the needling device 3000 may differ from a mixing spray region where the composition and the coolant are sprayed. In one example, the mixing spray region may be included in the needling region. In another example, the needling region may be included in the mixing spray region. In a further example, some of the needling region and some of the mixing spray region may overlap.

In the meantime, during the post-needling mixing spray method, for the target region having a relatively large area, needling may be performed on the entire target region, and the mixing spraying may be performed thereafter.

First, in the post-needling mixing spray method, a unit needling area and a unit mixing spray area may be defined.

In this case, the unit needling area may be determined by the shape of the needling structure 3100. In one example, when the needling device 3000 operates in a rolling manner, and the body part 3110 of the needling structure 3100 has a cylindrical shape, the unit needling area has the area of the side surface of the body part 3110. In other words, when the body part 3110 is rotated once while being in contact with the target region, needling is performed on the target region as much as the unit needling area. In another example, when the needling device 3000 operates in a stamping manner or an automatic punching manner, a portion of the body part 3110 of the needling structure 3100 that contacts with the target region may be determined as the unit needling area.

Furthermore, in this case, the unit mixing spray area may be defined as an area where the coolant and the composition are sprayed, with the mixing spray system 100 disposed at a predetermined distance from the target region and the nozzle 1500 directed toward the target region.

The target region may be set to be relatively larger than the unit needling area or unit mixing spray area. For example, the target region may be the entire face, the entire palm, the entire sole, or the entire neck, other than a localized area.

When the target region is relatively large, the target region may be divided into a plurality of regions having the unit needling area or the unit mixing spray area, thereby performing the post-needling mixing spray method.

However, the mixing spraying can be performed after needling on the entire target region.

The target region may be divided into a plurality of subregions. The plurality of subregions may be determined on the basis of the unit needling area or the unit mixing spray area. For example, the area of the plurality of subregions may be determined to be larger than the unit needling area or the unit mixing spray area. The plurality of subregions may be determined to be arranged alongside or may be determined to overlap with each other.

Referring to FIG. 5, the target region may be divided into a first region R1 and a second region R2 that are arranged alongside. In addition, the area of the first region R1 or the area of the second region R2 is larger than the unit needling area or the unit mixing spray area.

Needling is performed on the first region R1, and then on the second region R2. Once needling is performed on the first region R1 and the second region R2, the mixing spraying may be performed on the first region R1 lastly.

In this case, the time point at which the mixing spraying is performed may be within a preset time from the time point at which needling begins on the first region R1. In other words, the time point at which the flow regulating unit 1200 is opened, with the nozzle 1500 directed toward the first region R1, may be within a preset time from the time point at which the needle structure 3100 contacts with the first region R1.

In this case, the preset time may vary depending on the area of the target region. For example, when the target region is the palm, the preset time may be determined within 1 minute or within 30 seconds.

In the meantime, while needling is performed on the second region R2, the mixing spraying may also be performed on the first region R1.

As described above, the mixing spraying is performed after the needling on the entire target region because the holes formed in the skin surface by the needling device 3000 can be preserved without closing. In addition, compared to performing the mixing spraying after needling on each of a plurality of unit regions (for example, regions corresponding to the unit needling area or the unit mixing spray area), performing the mixing spraying after needling on the entire region can significantly save procedure time.

However, to maximize the effect of procedures, the mixing spraying is preferably performed after needling on each of the unit regions by dividing the target region into a plurality of unit regions.

In the meantime, the mixing spraying may also be performed simultaneously with needling. For example, at least one user may use the mixing spray system 100 to spray the composition and the coolant toward the target region while simultaneously forming holes in the target region using the needling device 3000. However, in this case, there is a problem that the coolant and the composition fail to reach the target region immediately when needling is performed on the target region by the needling device 3000. Therefore, the mixing spraying is preferably performed after needling.

The post-needling mixing spray method may further include a pre-cooling step. For example, before the step of forming the plurality of holes by contacting the needling structure 3100 of the needling device 3000 with the target region (S1300), the user can lower the temperature of the target region by spraying the coolant onto the target region using the coolant spraying device 1000. By lowering the temperature of the target region, pain that may be caused by the needling device 3000 can be relieved. For pre-cooling, the temperature of the target region may be controlled to a range of about −5 °C to about 20 °C. Although the composition may be sprayed together with the coolant during the pre-cooling step, the composition is preferably not sprayed.

The post-needling mixing spray method may further include a post-cooling step. For example, after the step of spraying the composition and the coolant onto the target region (S1500), the user can lower the temperature of the target region by spraying the coolant onto the target region using the coolant spraying device 1000. By lowering the temperature of the target region, pain caused by the needling device 3000 can be relieved. For post-cooling, the temperature of the target region may be controlled to a range of about −5 °C to about 20 °C. Although the composition may be sprayed together with the coolant during the post-cooling step, the composition is preferably not sprayed.

### Penetration Effect of Post-Needling Mixing Spraying

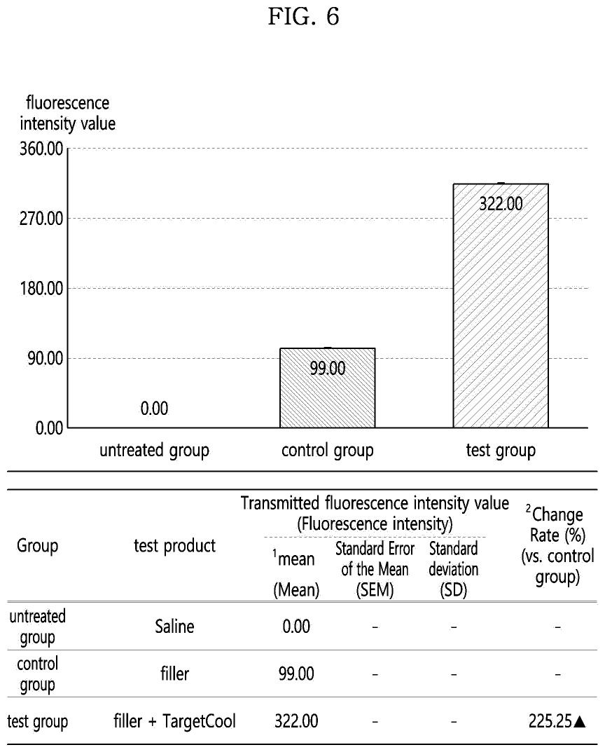

Hereinafter, the penetration effect of the post-needling mixing spraying is to be described with reference to FIGS. 6 to 14.

### Application Vs. Mixing Spraying

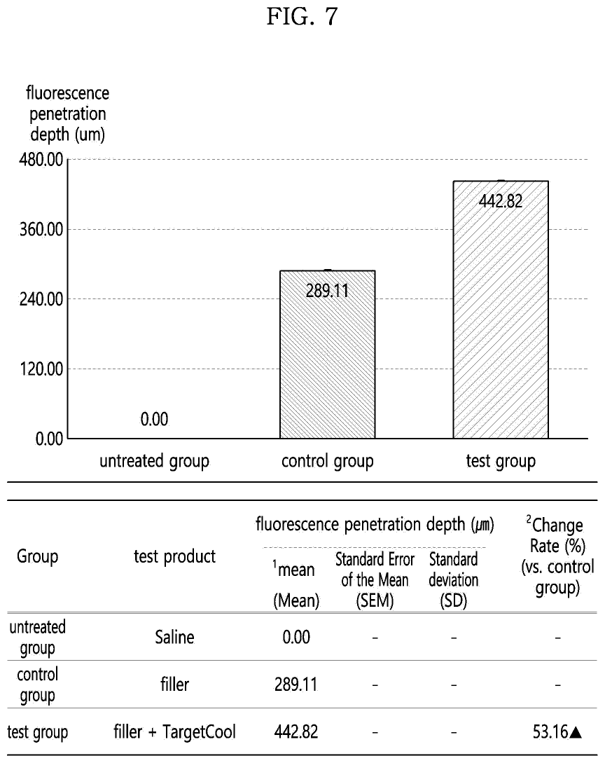

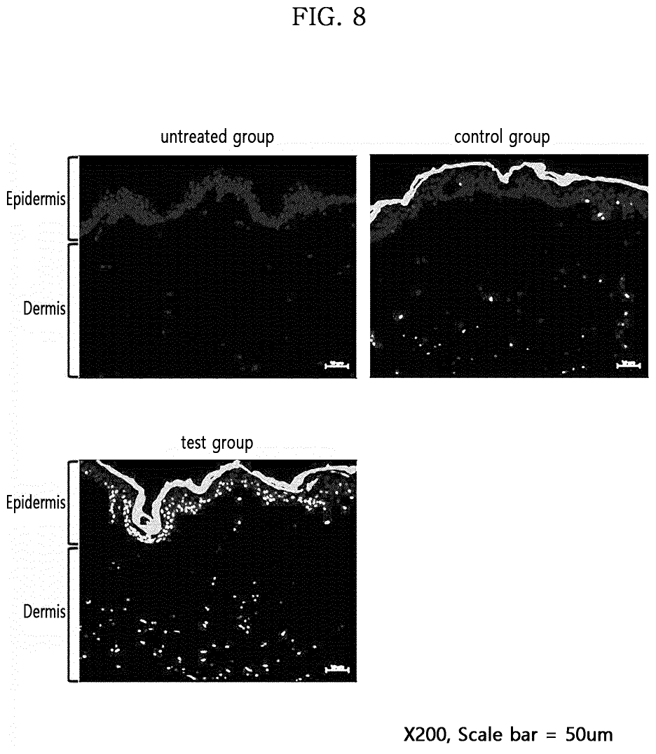

FIGS. 6 to 8 are diagrams showing the results of a first experiment to confirm the penetration effects of an application method and a mixing spray method in providing a composition to the skin. FIG. 6 is a diagram showing a graph and a table for comparing the penetration amount of a composition, FIG. 7 is a drawing showing a graph and a table for comparing the penetration depth of a composition in the same experiment, and FIG. 8 is a diagram showing the cross-sections of human-derived skin tissue used in the same experiment.

The objective of the first experiment was to compare the penetration effects of a method of applying a composition onto the skin surface and a method of spraying a composition and a coolant together onto the skin surface.

The first experiment was conducted to compare the penetration effects when applying saline solution onto the skin surface (untreated group), when applying a composition onto the skin surface (control group), and when spraying a composition and a coolant together onto the skin surface (test group).

The human-derived skin tissue served as a target where the composition was applied or sprayed.

In the untreated group, the human-derived skin tissue was cut into a predetermined size (3 cm×3 cm), followed by applying saline solution.

Then, 4′,6-diamidino-2-phenylindole (DAPI) staining and hematoxylin and eosin (H&E) staining were conducted on the human-derived skin tissue after 24 hours. Subsequently, fluorescence transmission images of the cross-sections of the human-derived skin tissue were taken using a fluorescence microscope, followed by confirming the fluorescence intensity and penetration depth.

In the control group, the human-derived skin tissue was cut into a predetermined size, followed by applying the composition. As the composition, a substance in which a fluorescent substance (fluorescein isothiocyanate, FITC) for confirming penetration effects was combined with a filler (a non-crosslinking hyaluronic acid (HA) filler product of TEOXANE) was used.

Then, DAPI staining and H&E staining were conducted on the human-derived skin tissue after 24 hours. Subsequently, fluorescence transmission images of the cross-sections of the human-derived skin tissue were taken using a fluorescence microscope, followed by confirming the fluorescence intensity and penetration depth.

In the test group, the human-derived skin tissue was cut into a predetermined size, followed by spraying the composition together with the coolant. When spraying the composition together with the coolant, the mixing spray system 100 described above was used. As the composition, a substance in which FITC for confirming penetration effects was combined with a filler was used. In addition, liquefied carbon dioxide gas served as the coolant.

Then, DAPI staining and H&E staining were conducted on the human-derived skin tissue after 24 hours. Subsequently, fluorescence transmission images of the cross-sections of the human-derived skin tissue were taken using a fluorescence microscope, followed by confirming the fluorescence intensity and penetration depth.

As a result of the first experiment, the penetration effect was significantly higher when spraying the composition together with the coolant using the mixing spray system 100 (test group) than when applying the composition (control group).

Referring to FIG. 6, the fluorescence intensity is 99.00 when applying the composition, whereas the fluorescence intensity is 322.00 when spraying the composition together with the coolant. Accordingly, the penetration effect is understood to be improved by about three times.

In addition, referring to FIG. 7, the fluorescence penetration depth is 289.11 μm when applying the composition, whereas the fluorescence penetration depth is 442.82 μm when spraying the composition together with the coolant. This demonstrates that the penetration depth differs by about 0.15 mm. Considering that the epidermis of the skin has a thickness in the range of 0.1 mm to 0.3 mm, it is expected that a depth difference of 0.15 mm may have a significant impact on whether the composition reaches the dermis.

Referring to the fluorescence intensity comparison data and fluorescence penetration depth comparison data from the first experiment, it is found that the penetration effect is significantly higher when spraying the composition together with the coolant than when applying the composition.

This can also be visually observed. Referring to FIG. 8, it is confirmed that, compared to the control group, a greater amount of the composition reached the dermis in the test group.

### Application vs. Needling vs. Mixing Spraying vs. Post-Needling Mixing Spraying

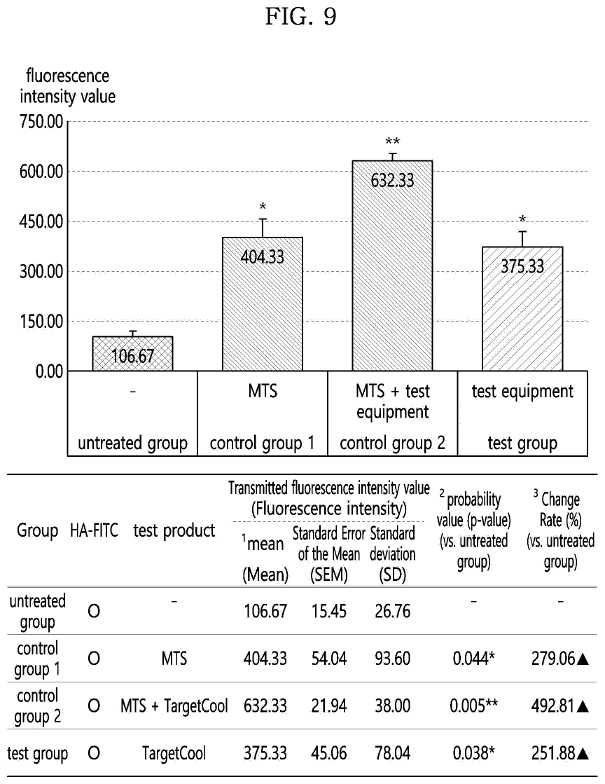

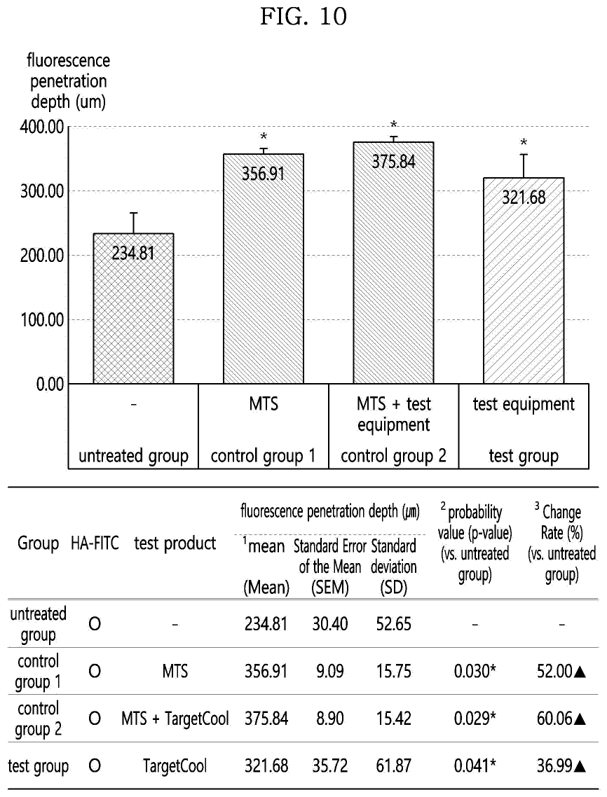

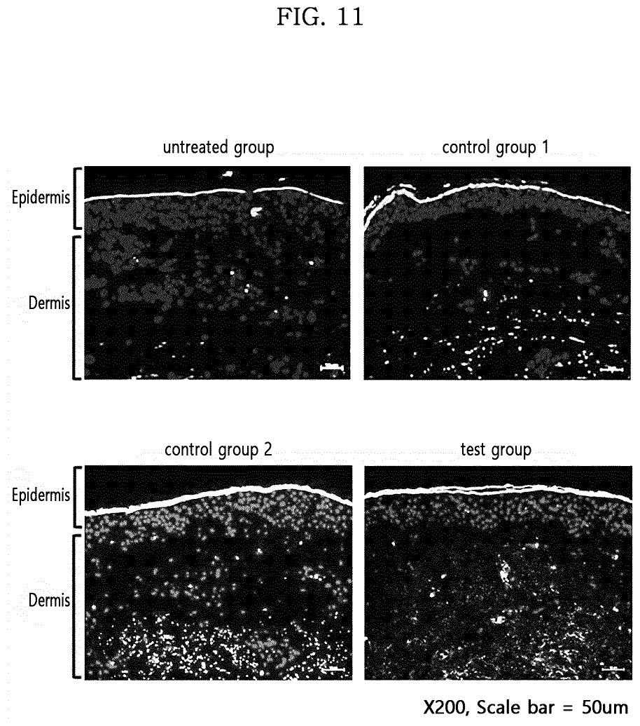

FIGS. 9 to 11 are diagrams showing the results of a second experiment to confirm the penetration effects of an application method, a mixing spray method, a needling method, and a post-needling mixing spray method in providing a composition to the skin. FIG. 9 is a diagram showing a graph and a table for comparing the penetration amount of a composition, FIG. 10 is a drawing showing a graph and a table for comparing the penetration depth of a composition in the same experiment, and FIG. 11 is a diagram showing the cross-sections of human-derived skin tissue used in the same experiment.

The objective of the second experiment was to compare the penetration effects of a method of applying a composition onto the skin surface, a method of applying a composition onto the skin surface and performing needling using a microneedle, a method of forming holes in the skin and spraying a composition together with a coolant, and a method of spraying a composition and a coolant together onto the skin surface.

The second experiment was conducted to compare the penetration effects when applying a composition onto the skin surface (untreated group), when using the needling device 3000 with a composition applied onto the skin surface (Control Group 1), when spraying a composition together with a coolant after using the needling device 3000 on the skin surface (Control Group 2), and when spraying a composition and a coolant together onto the skin surface (test group).

As the human-derived skin tissue serving as a test subject, facial skin tissue discarded after surgery was used. FITC combined with HA, namely, HA-FITC, served as the composition, and liquefied carbon dioxide gas served as the coolant.

In the untreated group, the human-derived skin tissue was cut into a predetermined size (2 cm×2 cm), followed by applying 500 μL of the composition.

Then, DAPI staining and H&E staining were conducted on the human-derived skin tissue after 24 hours. Subsequently, fluorescence transmission images of the cross-sections of the human-derived skin tissue were taken using a fluorescence microscope, followed by confirming the fluorescence intensity and penetration depth.

In Control Group 1, the human-derived skin tissue was cut into a predetermined size, followed by applying 500 μL of the composition and forming holes in the skin tissue.

When forming the holes in the skin tissue, the needling device 3000 operating in a rolling manner, as shown in FIG. 2, was used, and the needle length NL was 1.5 mm.

Then, DAPI staining and H&E staining were conducted on the human-derived skin tissue after 24 hours. Subsequently, fluorescence transmission images of the cross-sections of the human-derived skin tissue were taken using a fluorescence microscope, followed by confirming the fluorescence intensity and penetration depth.

In Control Group 2, the human-derived skin tissue was cut into a predetermined size, followed by forming holes and spraying 500 μL of the composition together with the coolant.

When forming the holes in the skin tissue, the needling device 3000 operating in a rolling manner, as shown in FIG. 2, was used, and the needle length NL was 1.5 mm. When spraying the composition together with the coolant, the mixing spray system 100 described above was used.

Then, DAPI staining and H&E staining were conducted on the human-derived skin tissue after 24 hours. Subsequently, fluorescence transmission images of the cross-sections of the human-derived skin tissue were taken using a fluorescence microscope, followed by confirming the fluorescence intensity and penetration depth.

In the test group, the human-derived skin tissue was cut into a predetermined size, followed by spraying 500 μL of the composition together with the coolant.

When spraying the composition together with the coolant, the mixing spray system 100 described above was used.

Then, DAPI staining and H&E staining were conducted on the human-derived skin tissue after 24 hours. Subsequently, fluorescence transmission images of the cross-sections of the human-derived skin tissue were taken using a fluorescence microscope, followed by confirming the fluorescence intensity and penetration depth.

The respective experiments on the untreated group, Control Group 1, Control Group 2, and the test group were conducted three times, and the experimental data was organized.

As a result of the second experiment, the penetration effect was significantly higher when forming the holes in the skin tissue and spraying the composition together with the coolant (Control Group 2) than when applying the composition (untreated group), when applying the composition and forming the holes (Control Group 1), and when spraying the composition together with the coolant (test group).

Referring to FIG. 9, the average fluorescence intensity in the untreated group was 106.67, the average fluorescence intensity in Control Group 1 was 404.33, and the average fluorescence intensity in the test group was 375.33, whereas the average fluorescence intensity in Control Group 2 was 623.33. Accordingly, the penetration effect when forming the holes in the skin tissue and spraying the composition together with the coolant may be understood to be higher than that when applying the composition and forming the holes by about 1.56 times and when spraying the composition together with the coolant by about 1.68 times. This means that, when a specific amount of the composition is intended to penetrate the skin for therapeutic or cosmetic purposes, the amount of the composition used differs by at least 1.5 times. Accordingly, considering the price of the composition marketed for therapeutic or cosmetic purposes, there is an effect of reducing the cost of medical or cosmetic services.

In the meantime, referring to FIG. 10, the fluorescence penetration depth in the untreated group was 234.81 μm, the fluorescence penetration depth in Control Group 1 was 356.91 μm, and the fluorescence penetration depth in the test group was 321.68 μm, whereas the fluorescence penetration depth in Control Group 2 was 375.84 μm.

Furthermore, referring to FIG. 11, it is confirmed that, compared to the untreated group, Control Group 1, and the test group, a greater amount of the composition reaches the dermis in Control Group 2.

As a result, compared to when using the needling device 3000 alone or when using the mixing spray system 100 alone, a greater amount of the composition penetrated the skin when using the needling device 3000 and the mixing spray system 100 together, and the depth to which the composition reached also increased.

In other words, through the second experiment, it was confirmed that when both the needling method and the mixing spray method, each sharing the concept of physically penetrating the epidermis of the skin, were performed together, the penetration effect was significantly improved without mutual interference.

### Application Vs. Needling Vs. Mixing Spraying Vs. Post-needling Mixing Spraying

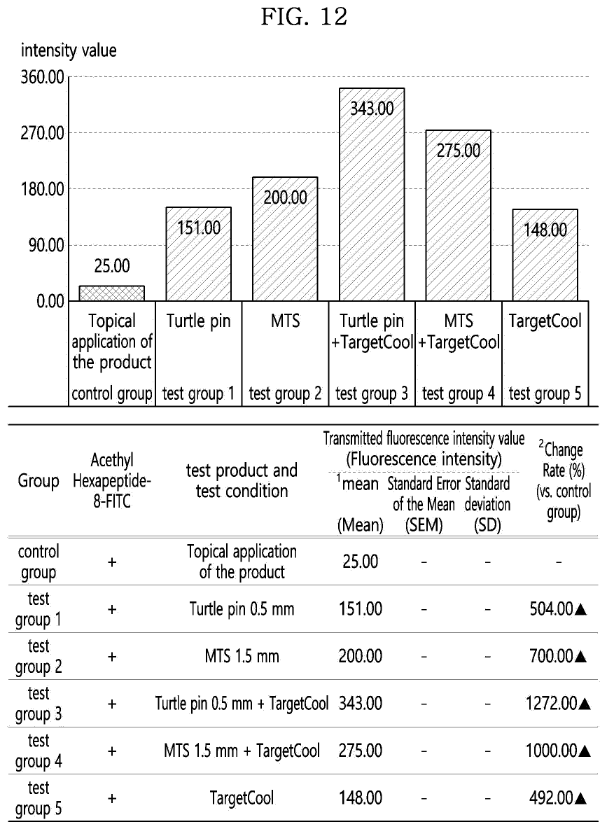

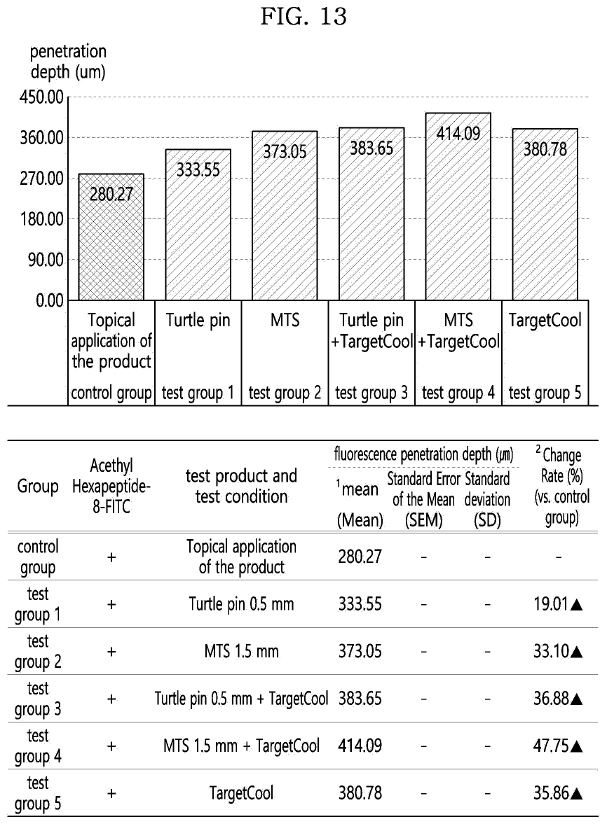

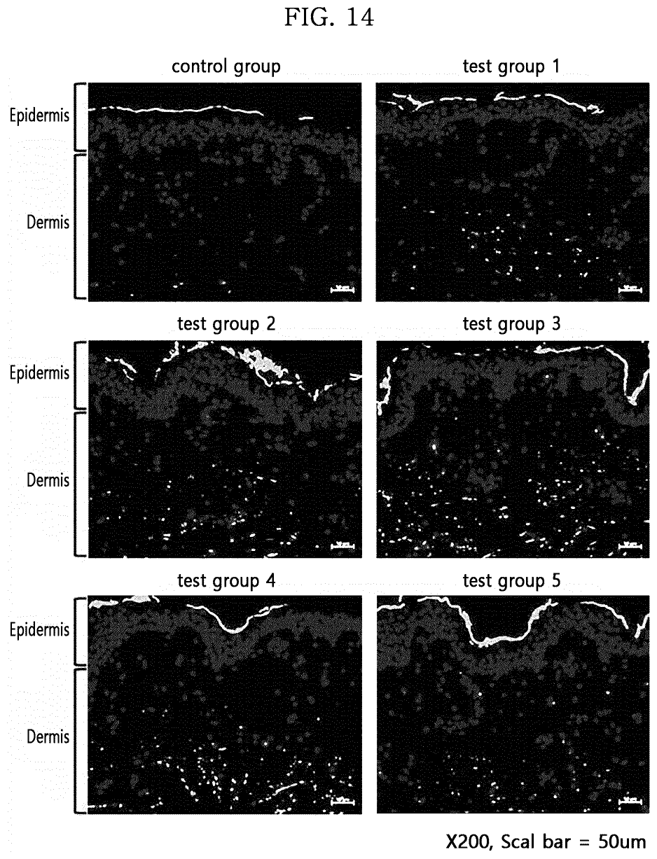

FIGS. 12 to 14 are diagrams showing the results of a third experiment to confirm the penetration effects of an application method, a mixing spray method, a needling method, and a post-needling mixing spray method in providing a composition to the skin. FIG. 12 is a diagram showing a graph and a table for comparing the penetration amount of a composition, FIG. 13 is a drawing showing a graph and a table for comparing the penetration depth of a composition in the same experiment, and FIG. 14 is a diagram showing the cross-sections of human-derived skin tissue used in the same experiment.

The objective of the third experiment was to compare the penetration effects of a method of applying a composition onto the skin surface, a method of applying a composition onto the skin surface and performing needling using a microneedle, a method of forming holes in the skin and spraying a composition together with a coolant, and a method of spraying a composition and a coolant together onto the skin surface. In addition, this experiment was to confirm the penetration effects when the needle length NL was different.

The third experiment was conducted to compare the penetration effects when applying a composition onto the skin surface (control group), when using a first needling device having a needle length NL of 0.5 mm, with a composition applied onto the skin surface (Test Group 1), when using a second needling device having a needle length NL of 1.5 mm, with a composition applied onto the skin surface (Test Group 2), when spraying a composition together with a coolant after using the first needling device on the skin surface (Test Group 3), when spraying a composition together with a coolant after using the second needling device on the skin surface (Test Group 4), and when spraying a composition and a coolant together onto the skin surface (Test Group 5).

As the human-derived skin tissue serving as a test subject, facial skin tissue discarded after surgery was used. A substance in which cross-linked HA combined with acetyl hexapeptide-8-FITC, in which acetyl hexapeptide-8 conjugated with a fluorescent substance, was used as the composition, and liquefied carbon dioxide gas was used as the coolant.

In the control group, the human-derived skin tissue was cut into a predetermined size (2 cm×2 cm), followed by applying 500 μL of the composition.

Then, DAPI staining and H&E staining were conducted on the human-derived skin tissue after 24 hours. Subsequently, fluorescence transmission images of the cross-sections of the human-derived skin tissue were taken using a fluorescence microscope, followed by confirming the fluorescence intensity and penetration depth.

In Test Group 1, the human-derived skin tissue was cut into a predetermined size, followed by applying 500 μL of the composition and forming holes in the skin tissue.

When forming the holes in the skin tissue, the first needling device operating in a rolling manner, as described with reference to FIG. 2, was used, and the needle length NL was 0.5 mm.

Then, DAPI staining and H&E staining were conducted on the human-derived skin tissue after 24 hours. Subsequently, fluorescence transmission images of the cross-sections of the human-derived skin tissue were taken using a fluorescence microscope, followed by confirming the fluorescence intensity and penetration depth.

In Test Group 2, the human-derived skin tissue was cut into a predetermined size, followed by applying 500 μL of the composition and forming holes in the skin tissue.

When forming the holes in the skin tissue, the second needling device operating in a rolling manner, as described with reference to FIG. 2, was used, and the needle length NL was 1.5 mm.

Then, DAPI staining and H&E staining were conducted on the human-derived skin tissue after 24 hours. Subsequently, fluorescence transmission images of the cross-sections of the human-derived skin tissue were taken using a fluorescence microscope, followed by confirming the fluorescence intensity and penetration depth.

In Test Group 3, the human-derived skin tissue was cut into a predetermined size, followed by forming holes and spraying 500 μL of the composition together with the coolant.

When forming the holes in the skin tissue, the first needling device described above was used. When spraying the composition together with the coolant, the mixing spray system 100 described above was used.

Then, DAPI staining and H&E staining were conducted on the human-derived skin tissue after 24 hours. Subsequently, fluorescence transmission images of the cross-sections of the human-derived skin tissue were taken using a fluorescence microscope, followed by confirming the fluorescence intensity and penetration depth.

In Test Group 4, the human-derived skin tissue was cut into a predetermined size, followed by forming holes and spraying 500 μL of the composition together with the coolant.

When forming the holes in the skin tissue, the second needling device described above was used. When spraying the composition together with the coolant, the mixing spray system 100 described above was used.

Then, DAPI staining and H&E staining were conducted on the human-derived skin tissue after 24 hours. Subsequently, fluorescence transmission images of the cross-sections of the human-derived skin tissue were taken using a fluorescence microscope, followed by confirming the fluorescence intensity and penetration depth.

In Test Group 5, the human-derived skin tissue was cut into a predetermined size, followed by spraying 500 μL of the composition together with the coolant.

When spraying the composition together with the coolant, the mixing spray system 100 described above was used.

Then, DAPI staining and H&E staining were conducted on the human-derived skin tissue after 24 hours. Subsequently, fluorescence transmission images of the cross-sections of the human-derived skin tissue were taken using a fluorescence microscope, followed by confirming the fluorescence intensity and penetration depth.

As a result of the third experiment, the penetration effect was significantly higher when forming the holes in the skin tissue and spraying the composition together with the coolant (Test Groups 3 and 4) than when applying the composition (control group), when applying the composition and forming the holes (Test Groups 1 and 2), or when spraying the composition together with the coolant (Test Group 5).

Referring to FIG. 12, the fluorescence intensity in the control group was 25.00, the fluorescence intensity in Test Group 1 was 151.00, and the fluorescence intensity in Test Group 5 was 148.00, whereas the fluorescence intensity in Test Group 3 was 343.00. Accordingly, the penetration effect when forming the holes in the skin tissue using the first needling device having a needle length NL of 0.5 mm and spraying the composition together with the coolant may be understood to be higher than that when applying the composition and forming the holes using the first needling device by about 2.27 times.

In addition, referring to FIG. 12, the fluorescence intensity in the control group was 25.00, the fluorescence intensity in Test Group 2 was 200.00, and the fluorescence intensity in Test Group 5 was 148.00, whereas the fluorescence intensity in Test Group 4 was 275.00. Accordingly, the penetration effect when forming the holes in the skin tissue using the second needling device having a needle length NL of 1.5 mm and spraying the composition together with the coolant may be understood to be higher than that when applying the composition and forming the holes using the second needling device by about 1.37 times.

This means that, as in the second experiment above, the cost of medical or cosmetic services can be reduced when supplying the composition using both the needling device 3000 and the mixing spray system 100, compared to when supplying the composition using the needling device 3000 alone or when supplying the composition using the mixing spray system 100 alone.

Furthermore, referring to FIG. 14, it is confirmed that, compared to the control group and Test Groups 1, 2, and 5, a greater amount of the composition reaches the dermis in Test Groups 3 and 4.

In the meantime, referring to FIG. 13, the fluorescence penetration depth in the control group was 280.27 μm, the fluorescence penetration depth in Test Group 1 was 333.55 μm, the fluorescence penetration depth in Test Group 2 was 373.05 μm, the fluorescence penetration depth in Test Group 3 was 383.65 μm, the fluorescence penetration depth in Test Group 4 was 414.09 μm, and the fluorescence penetration depth in Test Group 5 was 380.78 μm.

Considering Test Groups 1, 3, and 5, it was confirmed that the fluorescence penetration depth had a greater value when using the mixing spray system 100 alone than when using the first needling device having a needle length NL of 0.5 mm alone and, even when using the first needling device and the mixing spray system 100 together, the fluorescence penetration depth was not significantly improved compared to when using the mixing spray system 100 alone.

In the meantime, considering Test Groups 2, 4, and 5, it was confirmed that the fluorescence penetration depth was significantly improved when using the second needling device and the mixing spray system 100 together, compared to when using the second needling device having a needle length NL of 1.5 mm alone or when using the mixing spray system 100 alone.

Considering the above results, using the mixing spray system 100 may be understood to have an effect similar to that of using the needling device 3000 having a needle length NL of 0.5 mm, in terms of penetration depth.

Even through the third experiment, it was confirmed that, compared to when using the needling device 3000 alone or when using the mixing spray system 100 alone, a greater amount of the composition penetrated the skin when using the needling device 3000 and the mixing spray system 100 together, and the depth to which the composition reached also increased.

### Experimental Result Analysis

Considering that consistent results (for example, the penetration effect improved by about 1.5 times or more) were obtained despite using the needling devices 3000 having different needle lengths NLs in the third experiment and the types of substances used as the compositions differed from each other in the second and third experiments, it is expected that the penetration effect of the composition into the skin can be improved when using the needling method and the mixing spray method together in a complementary relationship, and in particular, the post-needling mixing spray method of forming holes in the target region and spraying the composition and the coolant can achieve a significant penetration effect of the composition.

### Post-Needling Freeze Spraying

Hereinabove, in describing the post-needling mixing spray method, it has been explained that the composition is sprayed together with the coolant so that the low-temperature composition reaches the skin surface.

When spraying the composition and the coolant together, the composition is introduced into a coolant spray stream in a liquid state, and most of the composition reaches the skin surface in the liquid state. In this case, when the temperature of the coolant spray stream is precisely controlled, the composition introduced as a liquid may be frozen and reach the skin surface in a solid state.

When the composition is frozen in the solid state and reaches the skin surface, the penetration effect may be improved compared to when the composition reaches the skin surface in the liquid state.

Hereinafter, a freeze spray method, in which a composition is frozen and enabled to reach the skin surface, is to be described. Furthermore, a post-needling freeze spray method, which is a combination of the needling method and the freeze spray method, is to be described.

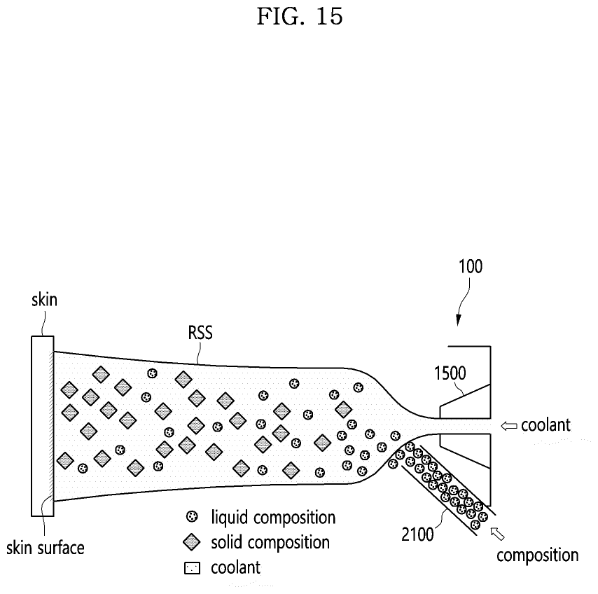

First, freeze spraying is to be described with reference to FIGS. 15 to 30 below.

In the process of developing the mixing spray system 100, the present applicant conducted an experiment to determine the degree to which the composition penetrates into the skin when spraying the composition and the coolant together, and confirmed that the composition effectively penetrated into the skin compared to conventional methods. As a result of conducting research into the reason for this, it was confirmed that the penetration effect improved not simply because the composition was sprayed together with the coolant, but because the composition was sprayed in a frozen state.

More specifically, while observing the process of spraying the composition and the coolant together in the mixing spray system 100, the present applicant confirmed that a part of the composition that was in a liquid state before spraying was frozen by the coolant and collided with the skin surface in a solid state.

FIG. 15 is a view illustrating how the composition is frozen and sprayed according to an embodiment. Referring to FIG. 15, when a coolant spray stream RSS is formed by the nozzle 1500, the composition is discharged in a liquid state from the composition guide 2100 and introduced into the coolant spray stream RSS. As the composition in a liquid state is sprayed along the coolant spray stream RSS, it transitions into solid frozen particles, and the frozen particles reach the skin surface.

The present applicant assumed that the fact that the composition is frozen by the coolant and becomes frozen particles is a factor affecting improvement of the penetration ability of the composition, and as described later, improved the mixing spray system 100 for freeze spraying while conducting experiments on freeze spraying.

### Regarding Freeze Spraying

Freeze spraying means colliding the composition in a frozen state on the skin surface so that the composition has a significant penetration effect on the skin.

The significant penetration effect means that the composition that penetrates into the skin is equal to or greater than a predetermined level. In addition, the significant penetration effect means that the composition penetrates into the skin deeply.

More specifically, the significant penetration effect means that when the composition is frozen and sprayed on the skin surface, the amount of composition reached below the epidermis of the skin is equal to or greater than a predetermined level compared to the amount of composition sprayed.

Alternatively, when the same amount of composition is mixed with the coolant sprayed at the same velocity and sprayed on the skin surface, the penetration effect may be significant when the amount of composition penetrated is larger than when the composition is sprayed on the skin surface in an unfrozen state.

Alternatively, the significant penetration effect means that when the composition is frozen and sprayed on the skin surface, at least a part of the composition reaches below the epidermis of the skin.

When the composition is applied to the skin surface by freeze spraying, the composition is expected to pass through the epidermis more easily. This is because when the composition is frozen and collides in the form of a solid composition with the skin surface, the impact force thereof is expected to be larger than that of a liquid composition.

Specifically, when liquid composition particles and solid composition particles with the same momentum (mass*velocity) each collide with the skin surface, the harder solid composition particles has a larger impact force due to their shorter collision time, and as a result, may more easily pass through the stratum corneum.

In addition, since the solid composition particles are crystalline solids, the collision area thereof with the skin surface may be smaller than that of the liquid composition particles. Therefore, the solid composition particles have a larger impact force per area and may pass through the stratum corneum more easily than the liquid composition particles.

The fact that the frozen composition passes through the stratum corneum may be understood that frozen particles of the composition form holes on the skin surface and penetrate into the stratum basale of the epidermis or the dermis therebelow. Since the frozen composition functions as a needle, the freeze spray method may be called an ice needling method.

### Freeze Spraying With Improved Penetration Effect

As described above, freeze spraying means colliding the composition in a frozen state with the skin surface so that the composition has a significant penetration effect on the skin.

Meanwhile, the present applicant conducted an experiment on freeze spraying as described later, and confirmed that when the degree to which the composition was frozen reached a predetermined level, the penetration effect of the composition improved compared to when spraying the composition in an unfrozen state.

More specifically, it was confirmed whether penetration of the composition improved when the temperature of the coolant spray stream was adjusted so that the composition was moved in a frozen state until it reaches the skin surface, and as a result, it was confirmed that the penetration effect of the composition improved. A first experiment related to this will be described later.

In addition, it was necessary to confirm how the penetration effect of the composition varies depending on the degree to which the composition is frozen when it reaches the skin surface. The present applicant conducted an experiment to confirm this along with a second experiment which will be described later, and before that, established an objective criteria for the degree to which the composition was frozen in freeze spraying.

Here, as a method of determining the degree to which the composition was frozen, a method of calculating and evaluating a freeze ratio when the mixing spray system 100 is viewed from the side (e.g., the left side or right side) was used. Here, the state in which the mixing spray system 100 is viewed from the side means a state in which the mixing spray system 100 is viewed from the left or right side when operated normally. For example, the image illustrated in FIG. 4 is an image of the mixing spray system 100 when viewed from the right side. The method of calculating the freeze ratio and the method of designing the mixing spray system 100 for controlling the freeze ratio will be described later.

First, when the mixing spray system 100 performing freeze spraying is viewed from the side, and the freeze ratio is 17% (within a measurement error of 5%, about 12% to about 22%), the penetration effect is higher than when the freeze ratio is substantially 0%. Therefore, when the composition is sprayed on the skin surface with a freeze ratio of 17%, the penetration effect of the composition is significant, and spraying with the corresponding freeze ratio may be understood as freeze spraying.

In addition, when the mixing spray system 100 performing freeze spraying is viewed from the side, and the freeze ratio is 5% (within a measurement error of 1%, about 4% to about 6%), the penetration effect is higher than when the freeze ratio is substantially 0%. Therefore, when the composition is sprayed on the skin surface with a freeze ratio is 5%, the penetration effect of the composition is significant, and spraying with the corresponding freeze ratio may be understood as freeze spraying.

In addition, it was confirmed that the penetration effect of the composition improved as the freeze ratio of the composition reached the skin surface increased. Specifically, the penetration effect improved in the following order: when the freeze ratio of the composition is 5% (within a measurement error of 1%, about 4% to about 6%), when the freeze ratio of the composition is 17% (within a measurement error of 5%, about 12% to about 22%), when the freeze ratio of the composition is 48% (within a measurement error of 3%, about 45% to about 51%), when the freeze ratio of the composition is 71% (within a measurement error of 3%, about 68% to about 74%), and when the freeze ratio of the composition is 100% (within a measurement error of 1%, about 99% to about 100%).

In other words, when the penetration effect is improved under the condition that the freeze ratio of the composition is 5%, a high penetration effect can be achieved even when the freeze ratio of the composition is equal to or greater than 5%. Therefore, the freeze ratio of the composition may be adjusted from 5% to 100%. Alternatively, the freeze ratio of the composition may be adjusted from 5% to 17%. Alternatively, the freeze ratio of the composition may be adjusted from 5% to 48%. Alternatively, the freeze ratio of the composition may be adjusted from 5% to 71%.

Considering the measurement error range, the freeze ratio of the composition may be adjusted from 2% to 100%. Alternatively, the freeze ratio of the composition may be adjusted from 2% to 22%. Alternatively, the freeze ratio of the composition may be adjusted from 2% to 51%. Alternatively, the freeze ratio of the composition may be adjusted from 2% to 74%.

Meanwhile, it was confirmed that when the skin surface was excessively cooled by the coolant or composition in the process of increasing the freeze ratio of the composition, the penetration effect of the composition decreased. More specifically, even when the freeze ratio of the composition reaching the skin surface is high, a decrease in the temperature of the skin surface may cause a penetration-impeding substance such as an ice film to be formed, thus impeding the composition from reaching the skin surface. In this case, the penetration effect of the composition may be decreased. Therefore, it is necessary to design the mixing spray system 100 in consideration of increasing the freeze ratio of the composition while maintaining the temperature of the skin surface equal to or greater than an appropriate temperature, and the specific design method will be described later.

As in the examples above, when the mixing spray system 100 performing freeze spraying is viewed from the side, the freeze ratio is calculated, and when the penetration effect is higher than when the freeze ratio is 0%, the composition is frozen and sprayed with a freeze ratio equal to or greater than the calculated freeze ratio. This may be understood as freeze spraying.

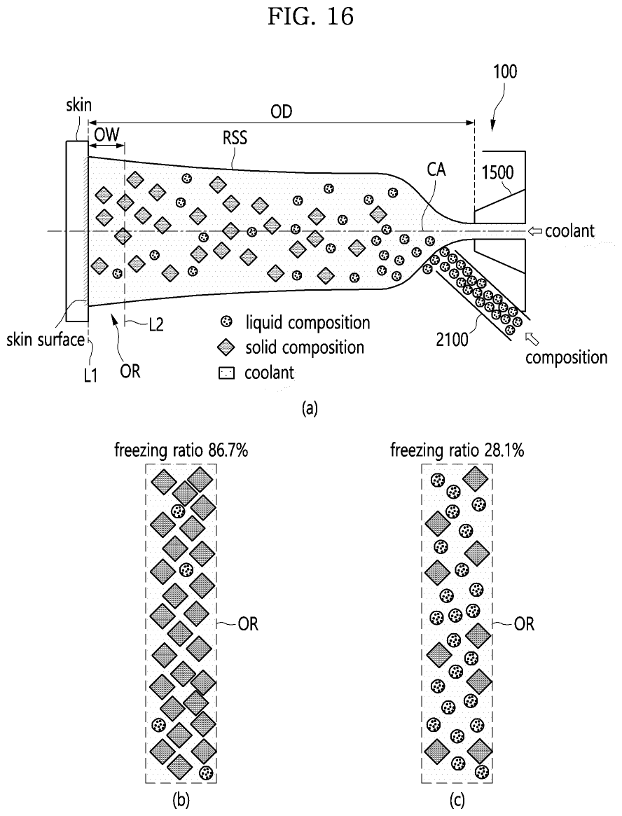

Hereinafter, the method of calculating the freeze ratio will be described with reference to FIGS. 16 to 19.

FIG. 16 is a view illustrating how the freeze ratio of the composition is determined using an observing region OR according to an embodiment. (a) of FIG. 3 illustrates that the observing region OR is specified based on the mixing spray system 100, and (b) of FIG. 3 and (c) of FIG. 3 respectively illustrate cases with different freeze ratios in the observing region OR.

### Calculation of Freeze Ratio of Composition in Observing Region

A high-speed camera may be used as a means of observing freeze spraying. Specifically, when the mixing spray system 100 sprays the coolant and the composition, the coolant spray stream RSS into which the composition introduced may be captured using the high-speed camera. An image or video captured with the high-speed camera clearly shows the form in which the composition is sprayed at a time point or over a time period, and the degree to which the composition is frozen may be determined through the image or video.

The observing region OR may be specified to observe freeze spraying. The observing region OR may refer to a region for determining the freeze ratio.

Specifying the observing region OR is to determine the freeze ratio of the composition reaching the skin surface. As the composition is sprayed together with the coolant, the composition may be frozen in any region within the coolant spray stream RSS. However, since freeze spraying is aimed at penetrating the composition into the skin, it is desirable to determine whether the composition that actually reaches the skin surface is frozen or determine at which ratio the composition is frozen. In other words, considering that the purpose of freeze spraying is to determine the freeze ratio that affects the penetration effect of the composition, it is desirable to observe a region adjacent to the skin surface during freeze spraying rather than observing the entire region where the composition can be frozen.

The observing region OR may be specified based on a position corresponding to the skin surface where the composition needs to be sprayed. For example, referring to (a) of FIG. 3, when the composition is sprayed on the skin surface by the above-described mixing spray system 100, a region spaced apart from an orifice of the nozzle 1500 of the mixing spray system 100 by a distance corresponding to a separation distance between the orifice of the nozzle 1500 and the skin surface when the mixing spray system 100 is viewed from the side may be specified as the observing region OR.

More specifically, when the mixing spray system 100 is viewed from the left or right side, a virtual first straight line L1 that is spaced apart from the orifice of the nozzle 1500 of the mixing spray system 100 by an observing distance OD and is orthogonal to a central axis CA of the nozzle 1500 may be determined. In addition, when the mixing spray system 100 is viewed from the left or right side, a second straight line L2 that is parallel to the first straight line L1 and is spaced apart from the first straight line L1 by an observing width OW in a direction toward the nozzle 1500 (or in a direction away from the nozzle 1500) may be determined. The observing region OR may be determined as a region between the first straight line L1 and the second straight line L2.

The observing distance OD may be determined based on a recommended spray distance. Alternatively, the observing distance OD may be determined based on an end of a distance maintenance unit, which will be described later. Alternatively, the observing distance OD may be determined based on the length of the distance maintenance unit.

Here, the recommended spray distance may refer to a distance at which the mixing spray system 100 needs to be positioned relative to the skin surface in the use of the mixing spray system 100. Specifically, the recommended spray distance may refer to a preferred straight-line distance between the nozzle 1500 and the skin surface when operating the mixing spray system 100 to spray the coolant and the composition on the skin surface. The recommended spray distance may be determined differently depending on the specifications of the components of the mixing spray system 100 (e.g., the internal pressure of the coolant container RC, the size of the orifice of the nozzle 1500, etc.).

The recommended spray distance is preferably longer than the length of an acceleration section required for the composition to accelerate. Specifically, the composition is frozen after being introduced into the coolant spray stream RSS, and the frozen composition is accelerated over a predetermined section. In order for frozen particles of the composition to be sufficiently accelerated, an acceleration section having a predetermined length in a direction parallel to the central axis CA of the nozzle 1500 is required. For example, in order for frozen particles of the composition to be sufficiently accelerated, an acceleration section of about 3 mm to about 5 mm or about 1 mm to about 10 mm is required. It is preferable that the above-described acceleration section is guaranteed based on a position where the composition is introduced into the coolant spray stream RSS. Therefore, the recommended spray distance may be set to be greater than the sum of the distance from the orifice of the nozzle 1500 to the position where the composition is introduced into the coolant spray stream RSS and the length of the above-described acceleration section based on the direction parallel to the central axis CA of the nozzle 1500.

The recommended spray distance is preferably longer than the length of the acceleration section required for the composition to accelerate.

In addition, the recommended spray distance is preferably longer than the length of a freeze section required for the composition to be introduced into the coolant spray stream RSS and frozen.

The recommended spray distance may be selected within a range of, for example, 3 mm to 100 mm. Meanwhile, since the recommended spray distance is a guide to the preferred spray distance, it may be presented as a range rather than a specific value. For example, the recommended spray distance may be presented as a range having a lower distance limit value and an upper distance limit value selected within a range of 3 mm to 100 mm.

The observing distance OD may be the same as the recommended spray distance. When the observing distance OD is the same as the recommended spray distance, a region where the skin surface is expected to be located may be specified as the observing region OR.

Meanwhile, the observing distance OD may be shorter than the recommended spray distance. When, the process of spraying the composition and the coolant on the skin surface is captured with the high-speed camera, it is difficult to determine the freeze ratio of the composition on the skin surface. Therefore, the freeze ratio determined at a predetermined distance from the skin surface may be estimated as the freeze ratio on the skin surface. In this case, the observing distance OD may be shorter than the recommended spray distance. Alternatively, when the mixing spray system 100 is viewed from the side, the observing region R may be determined by the first straight line L1 spaced from the skin surface by a first distance and the second straight line L2 parallel to the first straight line L1 and spaced apart from the first straight line L1 by the observing width OW.

The observing distance OD may be set to be greater than the recommended spray distance. In this case as well, the freeze ratio in the observing region OR may be estimated as the freeze ratio on the skin surface.

When the recommended spray distance is presented as a range, the observing distance OD may be included within the recommended spray distance range. The recommended spray distance range may be determined as a section within a range of 3 mm to 100 mm.

Meanwhile, the observing distance OD does not necessarily need to be determined based on the recommended spray distance, and may be determined arbitrarily.

The observing distance OD may be set to be greater than a minimum threshold distance value. In order for the composition to be introduced into the coolant spray stream RSS and frozen, heat exchange needs to occur between the coolant and the composition, so heat exchange takes a predetermined amount of time. Since the composition is moved by the coolant during heat exchange, the composition may not be frozen in a region closest to the nozzle 1500 through which the coolant is sprayed. Therefore, considering that the observing region OR is set to observe frozen particles, the observing distance OD may be set to be greater than the distance corresponding to the closest region from the nozzle 1500. For example, the observing distance OD may be set to be greater than a minimum threshold distance value determined within a range of 0 mm to 3 mm.

The observing width OW refers to a width of the observing region OR.

The observing width OW may be determined within a range of 1% to 50% of the observing distance OD. Alternatively, the observing width OW may be determined as the diameter of the coolant spray stream RSS at a position spaced apart by the observing distance OD from the orifice of the nozzle 1500. Alternatively, the observing width OW may be determined as an average diameter of the coolant spray stream RSS. Meanwhile, the observing width OW may not be specified as an arbitrary value, and the inside of the coolant spray stream RSS that can be seen with the naked eye may be the observing region OR.

The freeze ratio may be defined as a ratio of the number of solid composition particles to the sum of the number of solid composition particles and the number of liquid composition particles in a specific region. For example, referring to (b) of FIG. 3 or (c) of FIG. 3, the freeze ratio may be obtained by counting the number of solid composition particles and the number of liquid composition particles observed in the observing region OR and dividing the number of solid composition particles by the sum of the number of solid composition particles and the number of liquid composition particles.

Meanwhile, the freeze ratio may further take into account the number of gas composition particles. However, since it is difficult to calculate the number of gas composition particles, and the composition mostly exists in a solid or liquid state when the coolant and the composition are sprayed together, it is preferable that the freeze ratio does not take into account the number of gas composition particles.

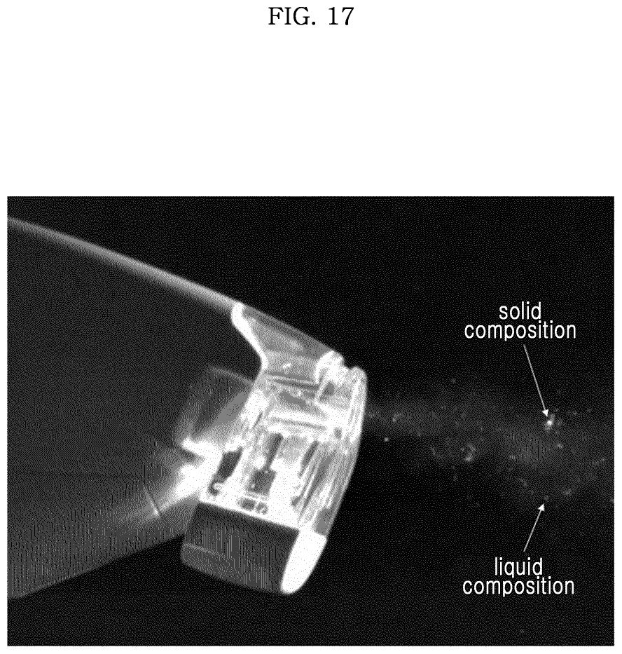

Hereinafter, a method of identifying solid composition particles in captured images will be described with reference to FIGS. 17 to 19.

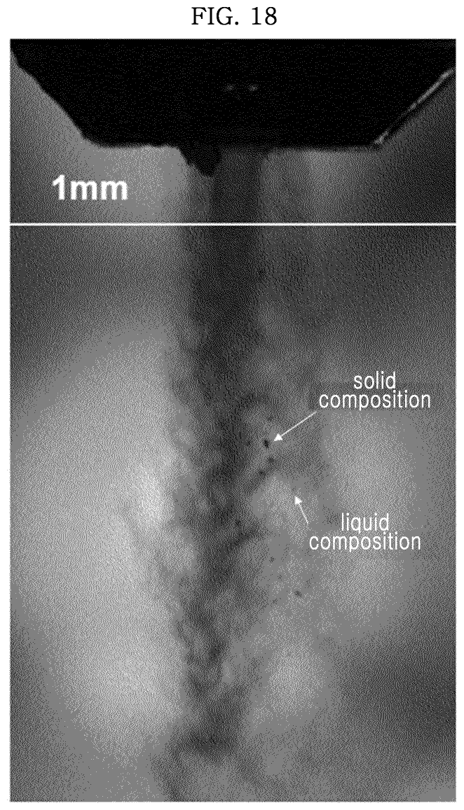

FIGS. 17 and 18 are images of a scene in which the liquid composition and the solid composition are sprayed according to an embodiment. FIG. 17 illustrates an image captured with the high-speed camera while light is emitted toward the mixing spaying system 100 that sprays the composition and the coolant. FIG. 18 illustrates an image of the mixing spray system 100 that sprays the composition and the coolant captured with the high-speed camera, in which light is emitted toward the high-speed camera and blocked by the composition.

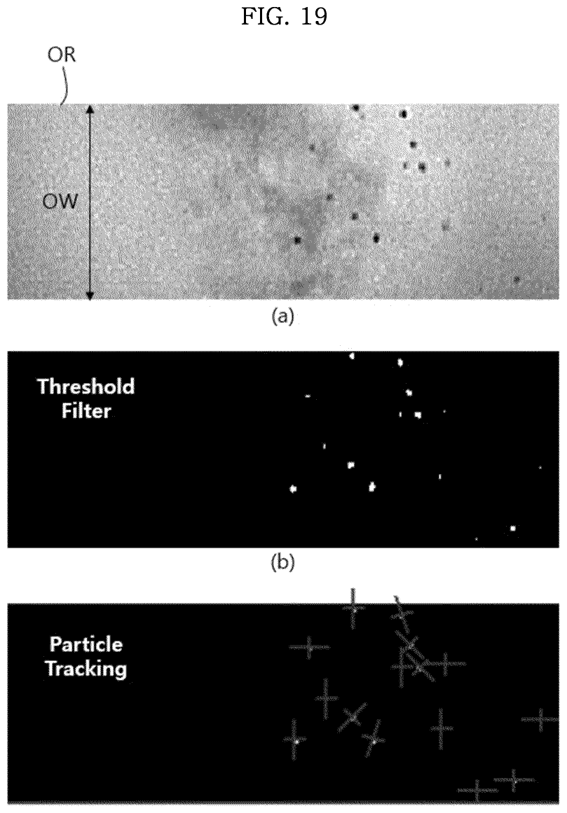

FIG. 19 is a view illustrating an image processing process for counting solid composition particles according to an embodiment. (a) of FIG. 19 illustrates an image of the observing region OR, (b) of FIG. 19 illustrates an image obtained by applying a threshold filter to the image of (a) of FIG. 19, and (c) of FIG. 19 illustrates tracking solid composition particles in the image of (b) of FIG. 19.

In calculating the freeze ratio, solid composition particles and liquid composition particles may be visually distinguished in high-speed camera images or videos (hereinafter referred to as “images, etc.”). Specifically, referring to FIG. 17, since the solid composition particles have a crystalline form, they reflect light and appear relatively bright.

Therefore, among the particles visibly identified in the images, etc. captured with the high-speed camera, particles that reflect light and appear bright may be specified as the solid composition particles, and particles that do not reflect light and appear dark may be specified as the liquid composition particles.

In addition, as illustrated in FIG. 18, the liquid composition particles and the solid composition particles may be distinguished based on the brightness of composition particles in images, etc. captured with the high-speed camera. Referring to FIG. 18, since the solid composition particles are darker than the liquid composition particles, the solid composition particles and the liquid composition particles may be identified based on the brightness with a predetermined level.

Referring to FIG. 19, the solid composition particles may be counted by separating the observing region OR from images, etc. captured with the high-speed camera, applying a filter that selects pixels with a brightness equal to or less than a predetermined level of brightness to the observing region OR, and tracking pixels that occupy more than a predetermined region.

In addition to the above-described methods for identifying solid composition particles, dynamic light scattering (DLS) with polarization analysis, time-resolved X-ray diffraction (TR-XRD), or in situ spectroscopy may be used.

Meanwhile, the freeze ratio may be defined as a ratio of the area of the solid composition particles to the sum of the area of the solid composition particles and the area of the liquid composition particles in a specified region.

According to an embodiment, the freeze ratio may be calculated in the following manner.

First, an image may be acquired using the high-speed camera while the composition and the coolant are sprayed together by the mixing spray system 100. Then, the observing region OR may be specified in the acquired image, and the freeze ratio may be calculated in the specified observing region OR.

Here, a plurality of images may be acquired.

For example, freeze spraying may be performed n times (n is a natural number of equal to or greater than 2) by the mixing spray system 100, and in an nth trial freeze spraying process, an nth trial image captured at a preset time after the start of spraying may be acquired. Thereafter, an nth trial freeze ratio may be calculated from the nth trial image, and the average of the first freeze ratio to the nth trial freeze ratio may be obtained as a final freeze ratio. Here, performing freeze spraying n times may be understood that spraying the composition and the coolant is performed n times in a state in which the same amount of coolant is stored at the same pressure in the coolant container RC and the same amount of composition is stored in the composition container CC.

For another example, the coolant and the composition may be sprayed from the mixing spray system 100 during a preset capture time, and at least two or more images may be acquired among images acquired during the capture time. The observing region OR may be specified and the freeze ratio may be calculated from each captured image, and the average of each freeze ratio for each captured image may be obtained as a final freeze ratio.

Meanwhile, when the plurality of images are acquired, in addition to averaging each freeze ratio obtained in each image, the freeze ratio may be calculated by adding the number of solid composition particles and the number of liquid composition particles counted in each image.

As described below, the penetration effect of the composition may vary depending on the freeze ratio. For example, as the freeze ratio increases, the penetration effect of the composition may increase.

Therefore, the mixing spray system 100 needs to be designed so that a freeze ratio at which a significant composition penetration effect is achieved is found and freeze spraying is performed with the corresponding freeze ratio.

In the above, it has been described that calculation of the freeze ratio in the observing region OR is understood as freeze spraying, but the technical idea of the present disclosure is not limited thereto.

For example, when the number of frozen particles in the observing region OR is equal to or greater than a preset threshold particle number when the composition and the coolant are sprayed together, this may be understood as freeze spraying.

Here, the threshold particle number is a value when the composition penetration effect on the skin is higher than when the number of frozen particles is substantially 0, and may be specified through experiments.

Meanwhile, a method of counting the number of frozen particles in the observing region OR is the same as the method of counting the number of frozen particles of the composition among the above-described freeze ratio calculation methods.

In the above, the method of specifying the observing region OR and calculating the freeze ratio when the mixing spray system 100 or spraying the composition and the coolant are viewed from the side has been described. However, the technical idea of the present disclosure is not limited thereto, and it is also possible to specify the observing region OR and calculate the freeze ratio when the mixing spray system 100 or spraying the composition and the coolant are viewed from above or below. Furthermore, it is also possible to calculate the freeze ratio by counting the solid composition particles and the liquid composition particles when viewing from two or more directions.

Likewise, when viewing from a direction other than the side or from two or more directions, a freeze ratio with a significant penetration effect may be calculated through experiments, and spraying the composition and the coolant with a freeze ratio equal to or greater than the corresponding freeze ratio may be understood as freeze spraying.

### Calculation of Freeze Ratio of Composition Passing Through Observing Line

The freeze ratio may be calculated based on an observing line OL as described below.

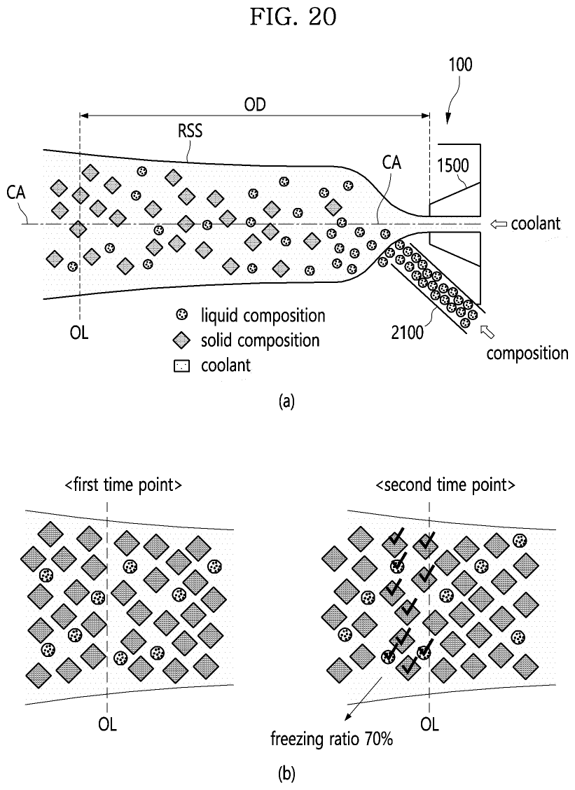

FIG. 20 is a view illustrating how the freeze ratio is calculated using the observing line OL according to an embodiment. (a) of FIG. 20 illustrates that the observing line OL is specified based on the mixing spray system 100, and (b) of FIG. 20 illustrates composition particles that passed through the observing line OL at first and second time points.

The observing line OL may be specified based on a position corresponding to the skin surface where the composition needs to be sprayed. For example, referring to (a) of FIG. 20, when the mixing spray system 100 that sprays the composition is viewed from the side, a straight line spaced a predetermined distance apart from the mixing spray system 100 may be specified as the observing line OL. More specifically, when the mixing spray system 100 is viewed from the left or right side, a virtual straight line that is spaced apart from the orifice of the nozzle 1500 of the mixing spray system 100 by an observing distance OD and is orthogonal to the central axis CA of the nozzle 1500 may be determined as the observing line OL.

Here, the observing distance OD is the same as that described in the observing region OR.

The freeze ratio may be calculated as a ratio of the number of frozen particles of the composition to the sum of the number of solid composition particles and the number of liquid composition particles passing through the observing line OL over a predetermined period of time. More specifically, referring to (b) of FIG. 20, the freeze ratio may be calculated in the following manner. First, an image may be acquired by capturing the side of the mixing spray system 100 that sprays the composition using the high-speed camera. Then, in the image, an image frame corresponding to a random first time point and an image frame corresponding to a second time point may be selected. Finally, in the selected image frames, the number of liquid composition particles and the number of solid composition particles that passed through the observing line OL from the first time point to the second time point may be counted.

The freeze ratio may be obtained as an average value of individual freeze ratios obtained for a plurality of images. Here, the plurality of images may include images obtained from each nth trial freeze spraying process. Also, here, the plurality of images may refer to images acquired in n time periods among images obtained by capturing spraying of the composition and the coolant from the mixing spray system 100 for a predetermined period of time.

As described in the observing region OR, the method of calculating the freeze ratio using the observing line OL may also assume that the mixing spray system 100 is viewed from above, below, or from two or more directions as well as from the side.

### Calculation of Freeze Ratio of Composition Sprayed on Observing Surface

The freeze ratio may be calculated using an observing surface as described below.

First, the observing surface may be placed at a position spaced apart by the observing distance OD in the mixing spray system 100, and the composition and the coolant may be sprayed on the observing surface while the high-speed camera captures the observing surface.

Thereafter, an image frame corresponding to a time point when the composition first reaches the observing surface or a time point after a predetermined period of time therefrom may be acquired from images captured by the high-speed camera.

The solid composition and the liquid composition may be distinguished based on color differences (or brightness differences) on the observing surface of the acquired image frame, and therefore, the area occupied by the solid composition and the area occupied by the liquid composition may be calculated based on the observing surface.

The freeze ratio may be calculated as a ratio of the area occupied by the solid composition to the sum of the area occupied by the solid composition and the area occupied by the liquid composition.

In the method of calculating the freeze ratio using the observing surface, a sensor that distinguishes between liquid and solid may be used in addition to the high-speed camera. For example, changes in temperature of an insulated plate may be monitored while spraying the composition and the coolant on the insulated plate, and the freeze ratio may be calculated based on the changes in temperature of the insulated plate or the amount of cooling energy (or heat) required to maintain the temperature and the latent heat absorbed by frozen particles when they transition into liquid. For another example, an electric touch sensor may be installed on the observing surface, and considering the capacitance upon contact of the liquid composition and the solid composition to the observing surface, the freeze ratio may be estimated based on the change in measured electrical signals.

In the method of calculating the freeze ratio using the observing surface, using the electric touch sensor

### Method of Generating Frozen Particles

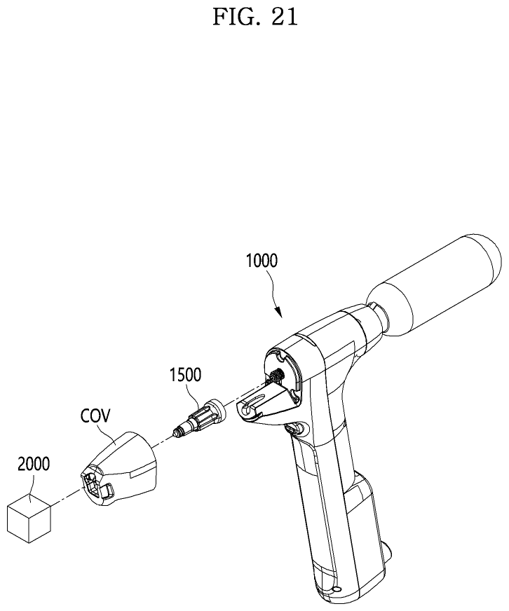

Hereinafter, a method for generating frozen particles by freezing the composition will be described with reference to FIGS. 21 to 24.

As described above, frozen particles of the composition may be generated using the mixing spray system 100. Specifically, the coolant spray stream RSS having a relatively low temperature may be formed by the coolant spray device 1000 of the mixing spray system 100, and frozen particles may be generated while the composition is supplied to the coolant spray stream RSS by the composition supply device 2000 of the mixing spray system 100.

FIG. 21 is a view illustrating a mixing spray system 100 according to an embodiment. Referring to FIG. 8, the mixing spray system 100 may include a coolant spray device 1000 and a composition supply device 2000. The composition supply device 2000 may be coupled to a nozzle 1500 of the coolant spray device 1000.

Meanwhile, as illustrated in FIG. 21, the mixing spray system 100 may further include a cover COV that covers the nozzle 1500 and supports the composition supply device 2000.

Hereinafter, for convenience of explanation, it will be described that the composition supply device 2000 is coupled to the nozzle 1500 of the coolant spray device 1000, but the technical idea of the present disclosure is not limited thereto. In addition to the nozzle 1500, the composition supply device 2000 may be coupled to the cover COV or a housing of the coolant spray device 1000. However, even in this case, a composition guide 2100 of the composition supply device 2000 needs to be disposed adjacent to the nozzle 1500 of the coolant spray device 1000 so that a composition discharged from the composition guide 2100 is introduced into a coolant spray stream RSS formed at the nozzle 1500.

### Detailed Component of Mixing Spray System

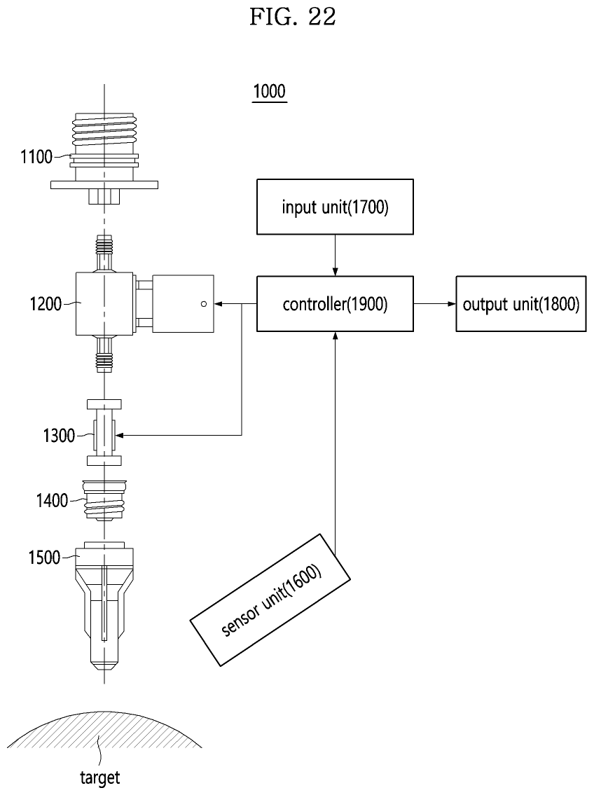

FIG. 22 is a view illustrating the components of a coolant spray device 1000 according to an embodiment. Referring to FIG. 22, the coolant spray device 1000 may include a container receiving unit 1100, a flow regulating unit 1200, a heat providing unit 1300, a nozzle coupling unit 1400, a nozzle 1500, a sensor unit 1600, an input unit 1700, an output unit 1800, and a controller 1900.

The container receiving unit 1100 may accommodate a coolant container RC. The container receiving unit 1100 is provided with a coolant receiving portion into which a coolant is introduced. The coolant receiving portion may be understood as a component including a flow path or hole for movement of the coolant.

For example, the coolant container RC may be provided as a portable cartridge, and the coolant container RC may be mounted on or detached from the container receiving unit 1100. When the coolant container RC is mounted on the container receiving unit 1100, the coolant in the coolant container RC may be moved to the coolant receiving portion of the container receiving unit 1100.

When the coolant container RC is provided as a cartridge and mounted on the container receiving unit 1100, a component for perforating a cartridge inlet to enable the coolant to come out of the cartridge and a component for sealing the perforated cartridge inlet to prevent the coolant from leaking to the outside may be required. Therefore, the component for perforating and the component for sealing may be disposed between the container receiving unit 1100 and the coolant container RC.

For another example, the coolant container RC may be provided as a tank that is difficult to carry, and the container receiving unit 1100 may be connected to the coolant container RC through a tube. The coolant in the coolant container RC may be moved to the coolant receiving portion of the container receiving unit 1100 through the tube.

The flow regulating unit 1200 may control movement of the coolant. For example, the flow regulating unit 1200 may include a valve, and the coolant may or may not be moved depending on whether the valve is opened or closed. Furthermore, the degree to which the coolant is moved may be determined depending on the opening ratio of the valve.

Here, the valve may be a solenoid valve, but the technical idea of the present disclosure is not limited thereto.

The container receiving unit 1100 and the flow regulating unit 1200 are fluidly connected to each other so that the coolant introduced into the coolant accommodation portion of the container receiving unit 1100 is moved to the flow regulating unit 1200. For example, the coolant receiving portion of the container receiving unit 1100 and a flow path of the flow regulating unit 1200 may be directly connected to each other. For another example, the coolant receiving portion of the container receiving unit 1100 and the flow regulating unit 1200 may be connected to each other through a conduit.

As described later, the coolant spray device 1000 may be equipped with a precise temperature control function that precisely controls the temperature of a spray region where the coolant is sprayed. The heat providing unit 1300 is one of the means for implementing the precise temperature control function and may provide heat to the coolant before the coolant is sprayed.

In order to achieve the purpose of precisely controlling the temperature of the spray region, the coolant spray device 1000 may heat the high-pressure/low-temperature coolant using the heat providing unit 1300 before the coolant is sprayed.

The heat providing unit 1300 may include a heat source and a heat transfer medium. The heat source is a component that produces heat and may, for example, include an element that uses a thermoelectric effect such as Peltier's effect. In this case, depending on the magnitude of power or current supplied to the heat source, the amount of heat energy produced by the heat source may vary. The heat transfer medium may provide heat produced by the heat source to the coolant. For example, the heat transfer medium may receive heat energy from the heat source and transfer the received heat energy to the coolant.

The heat transfer medium may be configured in various forms. For example, the heat source may be thermally coupled to the heat transfer medium, at least one flow path for movement of the coolant may be formed inside the heat transfer medium, and therefore, a component that maximizes the contact area (i.e., heat transfer area) between the heat transfer medium and the coolant may be provided.

The flow regulating unit 1200 and the heat providing unit 1300 may be fluidly connected to each other so that the coolant is moved from the flow regulating unit 1200 to the heat providing unit 1300. For example, the flow path of the flow regulating unit 1200 and a flow path of the heat providing unit 1300 may be directly connected to each other. For another example, the flow regulating unit 1200 and the heat providing unit 1300 may be connected to each other through a conduit.

The coolant may be sprayed through the nozzle 1500. The nozzle 1500 has a flow path formed therein for the coolant to flow. The flow path formed in the nozzle 1500 has narrower width at other end where the coolant is discharged than at one end where the coolant enters. The coolant is maintained at high pressure before the coolant is sprayed from the other end of the nozzle 1500, and the coolant sprayed from the other end of the nozzle 1500 is sprayed at high velocity while expanding adiabatically and is rapidly cooled. Here, as the pressure of the coolant introduced into the nozzle 1500 is higher, the temperature of the adiabatically expanded coolant has lower temperature and high velocity. For example, when the internal pressure of the coolant container RC is 50 bar, the pressure of the coolant introduced into the nozzle 1500 may be also close to 50 bar, and the temperature of the coolant sprayed from the nozzle 1500 may be about −50 °C. If the coolant of cryogenic temperature coolant, which has the high pressure required for high speed spray, is sprayed directly onto the skin, it may cause cell necrosis. In order to prevent skin damage such as cell necrosis, heat energy may be applied using the heat providing unit 1300 described above before the coolant is sprayed.

The nozzle 1500 is attachable to and detachable from the coolant spray device 1000. The nozzle coupling unit 1400 may be provided to attach or detach the nozzle 1500 to and from the coolant spray device 1000.

The composition supply device 2000 may be mounted on the nozzle 1500. For example, a part of the composition supply device 2000 may be coupled to the nozzle 1500 so that an output end of the composition guide 2100 of the composition supply device 2000 is disposed adjacent to an orifice of the nozzle 1500.

Meanwhile, the flow regulating unit 1200, the heat providing unit 1300, and the nozzle 1500 are fluidly connected to each other, and may be arranged in various ways. For example, the heat providing unit 1300 may be disposed between the flow regulating unit 1200 and the nozzle 1500 so that the coolant passes the flow regulating unit 1200 and reaches the heat providing unit 1300, and passes the heat providing unit 1300 and reaches the nozzle 1500. For another example, the flow regulating unit 1200 may be disposed between the heat providing unit 1300 and the nozzle 1500 so that the coolant passes the heat providing unit 1300 and reaches the flow regulating unit 1200, and passes the flow regulating unit 1200 and reaches the nozzle 1500.

The sensor unit 1600 may measure the temperature of the spray region where the coolant is sprayed. For example, the sensor unit 1600 may measure the temperature of the skin surface where the coolant is sprayed and provide measurement information to the controller 1900.

Meanwhile, the sensor unit 1600 may measure the temperature of a part of the components of the composition supply device 2000. For example, the sensor unit 1600 may measure the temperature of the composition guide 2100 or a mixing unit 2300 and provide measurement information to the controller 1900.

The input unit 1700 may receive a user's input. For example, the input unit 1700 may include at least one push button switch and provide a push input signal to the controller 1900 in response to the user's pressure on the switch, and the controller 1900 may control opening and closing of the flow regulating unit 1200 based on the push input signal. In addition, the input unit 1700 may include at least one rotary switch and provide a rotation input signal to the controller 1900 in response to the user's operation, and the controller 1900 may set a target temperature or target time based on the rotation input signal. Here, the target temperature refers to a temperature to be reached by adjusting the temperature of the spray region. In addition, the target time refers to a time during which spraying of the coolant needs to be maintained or a time during which the temperature of the spray region needs to be maintained at the target temperature. In addition, the user may use the input unit 1700 to set a target penetration depth of the composition. As described later, the mixing spray system 100 may control the size of frozen particles by controlling the heat applied to the coolant or the flow rate of the composition and thereby adjust the penetration depth.

The output unit 1800 may output an interface for use of the coolant spray device 1000 and various information to the user. For example, the output unit 1800 may include a display, and the output unit 1800 may output an interface for setting the above-described target temperature or target time through the display, and while the coolant spray device 1000 is operated, may output information such as a real-time temperature of the spray region measured by the sensor unit 1600 or a total time during which the coolant is sprayed.

The controller 1900 may control the components of the coolant spray device 1000. For example, the controller 1900 may control the temperature of the sprayed coolant by controlling the heat providing unit 1300, control the flow of the coolant by controlling the flow regulating unit 1200, and output specific information to the user through the output unit 1800.

The coolant spray device 1000 may be operated in the following manner.

First, the controller 1900 may set the target temperature and the target time. For example, the controller 1900 may provide an interface that guides the user to set the target temperature and the target time through the output unit 1800, receive a setting input signal in response to the user's operation through the input unit 1700, and set the target temperature and the target time based on the received setting input signal.

Thereafter, the controller 1900 may output a message indicating to the user that operation preparation is complete through the output unit 1800, receive a switch-on input signal in response to the user's operation through the input unit 1700, and enable the coolant to be sprayed based on the received switch-on input signal.

The controller 1900 may perform precise cooling on the spray region while the coolant is sprayed. For example, while the coolant is sprayed, the controller 1900 may acquire the real-time temperature of the spray region measured by the sensor unit 1600, and control the heat providing unit 1300 by comparing the obtained real-time temperature with the set target temperature. Specifically, when the obtained temperature value is lower than the target temperature, the controller 1900 may increase the heat energy applied to the coolant through the heat providing unit 1300, and when the obtained temperature value is higher than the target temperature, the controller 1900 may reduce the heat energy applied to the coolant through the heat providing unit 1300. Here, the controller 1900 may use proportional integral derivative (PID) control as a feedback control technology.

As precision cooling is performed by the controller 1900, the temperature of the spray region may be controlled within a predetermined error range based on the target temperature.

Meanwhile, the controller 1900 provide heat to the coolant using the heat providing unit 1300 regardless of the temperature of the spray region. For example, the controller 1900 may control the heat providing unit 1300 to provide heat energy per a predetermined unit time. In this case, temperature measurement for the spray region may not be performed.

Although not illustrated in FIG. 9, the coolant spray device 1000 may further include a distance maintenance unit. In spraying the coolant to the spray region, it is preferable that the distance between the target region and the coolant spray device 1000 is maintained constant. For example, it is preferable that the coolant and the composition are sprayed while the nozzle 1500 of the coolant spray device 1000 is located within a recommended spray distance range with respect to the target region.

In particular, when it is necessary to measure and monitor the temperature of the spray region in the coolant spray device 1000 (e.g., when feedback control is performed using the temperature of the target region, when the temperature of the target region decreases to equal to or lower than a safe temperature and the operation of the device stopped, or when the real-time temperature of the target region is output to the user), the temperature of the spray region needs to be accurately measured.

The distance maintenance unit may be located adjacent to the nozzle 1500. The distance maintenance unit may be connected to the housing of the coolant spray device 1000. The length of the distance maintenance unit may be designed so that the distance from the orifice of the nozzle 1500 to an end of the distance maintenance unit in a direction parallel to a central axis CA of the nozzle 1500 is within the recommended spray distance range. For example, the length of the distance maintenance unit may be determined based on a spray distance for maintaining the freeze ratio of the composition at equal to or greater than a predetermined value.

Meanwhile, the coolant spray device 1000 is not limited to the above-described embodiment, and any device or structure that performs the function of spraying coolant by coupling with the coolant container RC directly or indirectly through a tube may be regarded as the coolant spray device 1000 described in the present disclosure. For example, the coolant spray device 1000 may not heat the coolant, and therefore, the heat providing unit 1300 and the sensor unit 1600 may be omitted.

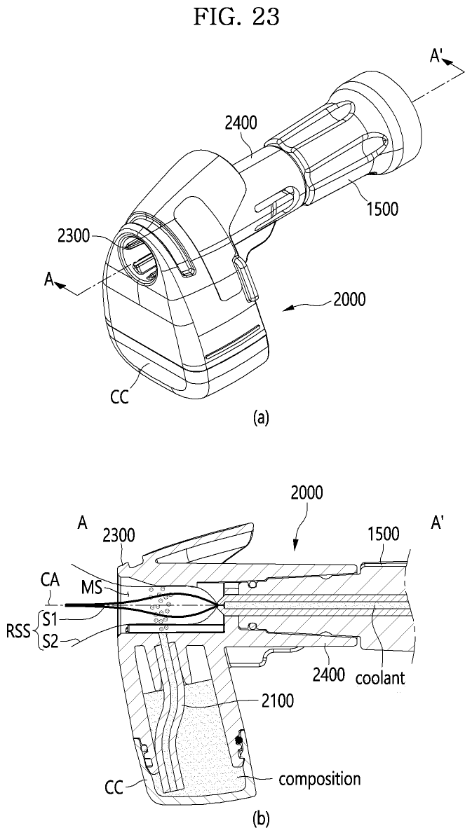

FIG. 23 is a view illustrating a composition supply device 2000 according to a first embodiment. (a) of FIG. 23 illustrates the composition supply device 2000 coupled to the nozzle 1500, and (b) of FIG. 23 illustrates a composition and a coolant being mixed and sprayed on a cross-section A-A′ with the composition supply device 2000 coupled to the nozzle 1500.

Referring to FIG. 23, the composition supply device 2000 may include a composition guide 2100, a mixing unit 2300, a composition container CC, and an coupling part 2400.

The composition guide 2100 serves to guide movement of the composition. For example, as illustrated in (b) of FIG. 23, the composition guide 2100 may fluidly connect the composition container CC and the mixing unit 2300, and the composition stored in the composition container CC may be moved to the mixing unit 2300 through the composition guide 2100. The composition guide 2100 may be implemented in the form of a tube. The composition guide 2100 may include an input end through which the composition is introduced and an output end through which the composition is discharged.

The mixing unit 2300 provides a mixing space MS where the composition and the coolant are mixed. As illustrated in (b) of FIG. 23, the mixing unit 2300 has an inner surface defining the mixing space MS. The output end of the composition guide 2100 may be located on the inner surface of the mixing unit 2300. The mixing space MS may be fluidly connected to the nozzle 1500 of the coolant spray device 1000, and when the coolant is sprayed from the nozzle 1500, a coolant spray stream RSS may be formed in the mixing space MS.

The coolant spray stream RSS may be divided into a main stream S1 and a sub-stream S2. The main stream S1 may refer to a region where the coolant is sprayed relatively strongly, and the sub-stream S2 may refer to a region where the coolant is sprayed relatively weakly. Alternatively, the main stream S1 may refer to a region where the density of the coolant is relatively high, and the sub-stream S2 may refer to a region where the density of the coolant is relatively low.

The main stream S1 and the sub-stream S2 may be divided based on the central axis CA of the nozzle 1500. For example, when the coolant spray stream RSS is cut perpendicularly to the central axis CA of the nozzle 1500, the main stream S1 may locate within a boundary distance from the central axis CA of the nozzle 1500, and the sub-stream S2 may locate outside the boundary distance from the central axis CA of the nozzle 1500. The boundary distance may vary depending on the distance from an end of the nozzle 1500, and the internal pressure of a coolant container RC and the size of the orifice of the nozzle 1500. For another example, a region where the temperature of the coolant is equal to or lower than a threshold temperature in the coolant spray stream RSS may be the main stream S1, and the remaining region may be the sub-stream S2. For another example, a region where the average velocity of the coolant is equal to or greater than a threshold velocity in the coolant spray stream RSS may be the main stream S1, and the remaining region may be the sub-stream S2. For another example, a region where the density of the coolant is equal to or greater than a threshold density in the coolant spray stream RSS may be the main stream S1, and the remaining region may be the sub-stream S2.

Meanwhile, it is important that the composition is introduced into the main stream S1 of the coolant spray stream RSS. Because the velocity of the coolant is faster and the temperature thereof is lower in the main stream S1 than in the sub-stream S2, the temperature of the composition becomes lower and the spray speed of the composition is also increases when the composition meets coolant particles and is sprayed in the main stream S1 rather than when the composition meets coolant particles and is sprayed in the sub-stream S2 spray speed.

The composition container CC may store the composition therein. The composition container CC may have an injection port through which the composition is injected. The composition container CC may have a ventilation hole through which external air is introduced.

The coupling part 2400 refers to a part coupled to the nozzle 1500 in the composition supply device 2000. For example, the coupling part 2400 may include a hook coupling member, a screw coupling member, or a force-fitting coupling member, and may be coupled to and fixed to one region of the nozzle 1500.

When the coolant spray stream RSS is formed in the mixing space MS, the coolant is sprayed adjacent to the output end of the composition guide 2100, and a negative pressure is formed at the output end of the composition guide 2100 according to Bernoulli's principle. Since the composition container CC has the ventilation hole, the internal pressure thereof is maintained at atmospheric pressure. Therefore, the composition stored in the composition container CC is moved to the output end of the composition guide 2100 where a lower pressure is formed, and is consequently introduced into the coolant spray stream RSS.

Meanwhile, a guide plate for moving the composition to the main stream S1 of the coolant spray stream RSS may be installed in the mixing unit 2300. The guide plate includes a surface having a preset length. A first end of the guide plate may be disposed adjacent to the output end of the composition guide 2100, and a second end of the guide plate may be disposed adjacent to the main stream S1. Therefore, the composition introduced through the composition guide 2100 may be moved along the guide plate and reach the main stream S1.

The components of the composition supply device 2000 may be manufactured integrally. Alternatively, at least parts of the components of the composition supply device 2000 may be manufactured separately and connected to each other.

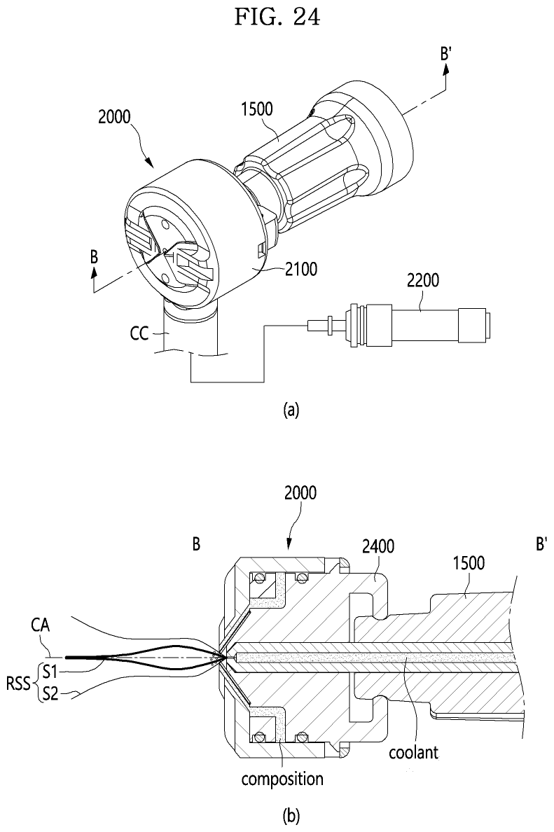

FIG. 24 is a view illustrating a composition supply device 2000 according to a second embodiment. (a) of FIG. 24 illustrates the composition supply device 2000 coupled to the nozzle 1500, and (b) of FIG. 24 illustrates a composition and a coolant being mixed and sprayed on a cross-section B-B′ with the composition supply device 2000 coupled to the nozzle 1500.

Referring to FIG. 24, the composition supply device 2000 may include a composition guide 2100, a composition container CC, an actuator 2200, and a coupling part 2400.

The composition guide 2100 is a component that receives the composition from the composition container CC and supply the composition to a coolant spray stream RSS. The composition guide 2100 may include an input end through which the composition is introduced and an output end through which the composition is discharged.

Referring to (b) of FIG. 24, a composition flow path through which the composition is moved is formed inside the composition guide 2100, and the output end of the composition guide 2100 may be disposed adjacent to the orifice of the nozzle 1500. Specifically, the output end of the composition guide 2100 may be disposed to be spaced apart from the orifice of the nozzle 1500 by a first distance in a direction parallel to the central axis CA of the nozzle 1500 and by a second distance in a direction orthogonal to the central axis CA of the nozzle 1500. Here, the output end of the composition guide 2100 may be located to make contact with a main stream S1 of the coolant spray stream RSS formed by the nozzle 1500. For example, the first distance and the second distance may be determined based on the boundary of the main stream S1 and a sub-stream S2 of the coolant spray stream RSS. The positional relationship between the output end of the composition guide 2100 and the nozzle 1500 will be described later.

The composition container CC is a component that stores the composition therein. The composition container CC may have an injection port through which the composition is injected. The composition container CC may be fluidly connected to the actuator 2200. The composition inside the composition container CC may be pressurized by the actuator 2200 and moved to the composition guide 2100.

The actuator 2200 may be configured to supply a fluid to a mixing unit 2300 at a preset flow rate. The actuator 2200 may include, for example, a piston and an electric motor, and may pressurize the fluid by receiving power and moving the piston.

Since the coupling part 2400 is the same as those described above, a description thereof will be omitted.

Referring to (b) of FIG. 24, when the coolant spray stream RSS is formed by the coolant spray device 1000, the actuator 2200 of the composition supply device 2000 may be operated to pressurize the composition in the composition container CC, causing the composition to be introduced into the coolant spray stream RSS through the composition guide 2100. Here, the flow regulating unit 1200 and the actuator 2200 may be controlled by the controller 1900. For example, the flow regulating unit 1200 may be operated first to spray the coolant, and then the actuator 2200 may be operated to discharge the composition. For another example, the flow regulating unit 1200 and the actuator 2200 may be operated simultaneously.

In the above, it has been described that the coolant spray device 1000 and the composition supply device 2000 are manufactured separately and coupled to each other, but the technical idea of the present disclosure is not limited thereto. For example, a part of the components of the composition supply device 2000 may be mounted on the coolant spray device 1000, and a part of the components of the coolant spray device 1000 may be implemented in the composition supply device 2000. Specifically, the composition supply device 2000 may include a component that performs the function of the nozzle 1500, and the composition supply device 2000 may be coupled to the nozzle coupling unit 1400 of the coolant spray device 1000.

As described above, the coolant spray stream RSS may be formed by the coolant spray device 1000, and the composition may be introduced into the coolant spray stream RSS by the composition supply device 2000. The composition introduced into the coolant spray stream RSS may be broken into microparticles in the coolant spray stream RSS and frozen by heat exchange with the coolant having a relatively low temperature. Here, the size of the microparticles may be about 10 μm to about 300 μm. Alternatively, the size of the microparticles may be about 10 μm to about 100 μm.

However, as will be described later, depending on the design method and control method of the mixing spray system 100, the composition may or may not be frozen, and the freeze ratio may vary.

### Factors Affecting Frozen Particle Generation

First, factors involved in whether and at what ratio the composition is frozen may include the temperature of the coolant spray stream RSS, composition flow rate, and composition inflow position.

As the temperature coolant spray stream RSS decreases, the composition may be frozen easily. Here, the temperature of the coolant spray stream RSS may refer to a temperature measured at a point within the coolant spray stream RSS (e.g., the temperature of a point spaced a predetermined distance apart from the orifice of the nozzle 1500). Alternatively, the temperature of the coolant spray stream RSS may refer to an average temperature of at least one region of the coolant spray stream RSS. As the temperature of coolant particles that exchange heat with the composition decreases, the temperature of the composition may decrease, thereby increasing the probability of freezing the composition. Furthermore, as the temperature of coolant particles decreases, the freeze ratio of the composition may increase.

Meanwhile, excessively lowering the temperature of the coolant spray stream RSS to freeze the composition may cause pain or irreversible damage to the skin. Therefore, the temperature of the coolant spray stream RSS needs to be controlled so that the composition does not cause pain or damage to the skin when frozen.

In addition, when the temperature of the coolant spray stream RSS excessively decreases, moisture on the skin surface may be frozen, forming substances that impede penetration, such as an ice film, and the composition may not reach the skin surface and bounce off the ice film.

The temperature of the coolant spray stream RSS may vary depending on the pressure before the coolant is sprayed from the nozzle 1500. For example, as the pressure of the coolant before the coolant is sprayed from the nozzle 1500 increases, the temperature of the coolant spray stream RSS may decrease. This is because as the coolant pressure before spraying increases, a difference with the coolant pressure after spraying (atmospheric pressure) increases, and temperature drop increases accordingly.

The coolant pressure before spraying may be substantially the same as the internal pressure of the coolant container RC. In other words, as the internal pressure of the coolant container RC increases, the coolant pressure before spraying may increase and the temperature of the coolant spray stream RSS may decrease.

Therefore, the coolant container RC with high internal pressure may be used to freeze the composition or increase the freeze ratio of the composition. Alternatively, the coolant container RC may be heated to increase the pressure of the coolant container RC.

Meanwhile, in order to increase the coolant pressure before spraying, the coolant spray device 1000 may further include a compressor. For example, the compressor may be disposed between the nozzle 1500 and the flow regulating unit 1200 or between the flow regulating unit 1200 and the container receiving unit 1100 to increase the pressure of the coolant.

The temperature of the coolant spray stream RSS may vary depending on the degree to which the coolant is heated before being sprayed from the nozzle 1500. As the amount of coolant heated increases, the temperature of the coolant spray stream RSS may increase. This is because the coolant temperature before spraying increases and the gas ratio in the coolant increases, reducing the amount of coolant that expands.

The amount of coolant heated may be adjusted by the heat providing unit 1300 of the coolant spray device 1000. For example, when the heat providing unit 1300 is a thermoelectric element that receives power and produces heat energy, the heat energy per unit time supplied from the heat providing unit 1300 to the coolant may be adjusted by adjusting the power applied to the heat providing unit 1300.

The amount of coolant heated may be controlled so that the temperature of the skin surface is equal to or greater than a minimum temperature that causes skin damage or pain. To this end, the controller 1900 of the coolant spray device 1000 may perform feedback control using a difference between the real-time temperature of the target region and the minimum temperature.

The temperature of the coolant spray stream RSS may be controlled using a method of controlling the coolant pressure before spraying, a method of controlling the heat energy applied to the coolant, or a combination thereof.

As the composition flow rate decreases, the composition may be frozen easily. The composition flow rate refers to an amount of composition that is introduced into the coolant spray stream RSS per unit time. Alternatively, the composition flow rate refers to an amount of composition that is supplied from the composition guide 2100 per unit time.

Since the composition introduced into the coolant spray stream RSS receives cooling energy (the temperature thereof decreases through heat exchange) by the coolant spray stream RSS, the cooling energy that the composition receives per unit mass decreases as the composition flow rate increases, so the composition may not be frozen or the freeze ratio may be reduced.

Meanwhile, as the composition flow rate decreases, the total amount of coolant used to spray a specific amount of composition to the target region may increase. That is, when the composition flow rate excessively decreases, the total amount of coolant used to spray the specific amount of composition may excessively increase, increasing the cost and time required for freeze spraying. Alternatively, when the composition flow rate excessively decreases and a predetermined amount of coolant is used, the amount of the composition sprayed may excessively decrease, thereby reducing the amount of composition penetrating into the skin.

Therefore, the composition flow rate needs to be controlled so that the composition is frozen and the amount of composition supplied is equal to or greater than a predetermined level during a predetermined treatment time.

The composition flow rate may be adjusted using the actuator 2200. For example, when supplying the composition to the composition guide 2100, the pressure applied to the composition may be controlled to be constant using the actuator 2200, and the composition flow rate may be controlled to be constant depending on the magnitude of the pressure. As the pressure applied to the composition increases, the composition flow rate increases, and as the pressure applied to the composition decreases, the composition flow rate decreases.

When the composition guide 2100 includes a conduit, the composition flow rate may be adjusted depending on the width of the conduit. Even when the composition is supplied from the composition container CC to the composition guide 2100 through the conduit, the composition flow rate may be adjusted depending on the width of the conduit. In other words, the composition flow rate may be determined depending on the width of the conduit designed when manufacturing the conduit. As the width of the conduit increases, the composition flow rate increases, and as the width of the conduit decreases, the composition flow rate decreases.

The composition flow rate may be controlled by using the actuator 2200, a method of designing the width of the conduit of the composition guide 2100 or the conduit connected to the composition guide 2100, or a combination thereof.

The closer the composition inflow position is to the nozzle 1500, the better the composition may be frozen. When spraying the composition and the coolant onto the skin surface using the mixing spray system 100, the composition is cooled by the coolant spray stream RSS from the position where it inflows into the coolant spray stream RSS until it reaches the skin surface.

In other words, as the distance from the position where the composition is introduced to the skin surface increases, the time it takes for the composition to be cooled by the coolant spray stream RSS increases, increasing the possibility of freezing or the freeze ratio of the composition. Alternatively, as the distance from the position where the composition is introduced to the orifice of the nozzle 1500 decreases, the time it takes for the composition to be cooled by the coolant spray stream RSS increases, increasing the possibility of freezing or the freeze ratio of the composition.

However, the composition inflow position may be understood as the position of the output end of the composition guide 2100, and when the output end of the composition guide 2100 is disposed excessively close to the orifice of the nozzle 1500, the coolant sprayed from the nozzle 1500 may collide with the composition guide 2100, forming turbulence. Considering stable spraying of the composition and the coolant and deceleration of the coolant spray stream RCC due to turbulence, it is preferable that no turbulence is formed.

The output end of the composition guide 2100 may be disposed to be spaced apart from the orifice of the nozzle 1500 by the first distance in a direction parallel to the central axis CA of the nozzle 1500 and by the second distance in a direction orthogonal to the central axis CA of the nozzle 1500.

The first distance may be within about 20 mm. The first distance may be, for example, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm.

The second distance may be within about 10 mm. The second distance may be, for example, 0.1 mm, 0.2 mm, 0.3 mm, 0.4 mm, 0.5 mm, 0.6 mm, 0.7 mm, 0.8 mm, 0.9 mm, or 10 mm. As described above, the first distance and the second distance decrease, the composition may be frozen easily, but when they excessively decrease, the problem of forming turbulence may occur.

For example, the composition inflow position may be determined as a position that is spaced a predetermined distance apart from the nozzle 1500 and corresponds to the edge of the main stream S1 of the coolant spray stream RSS formed by the nozzle 1500. In this case, the first distance and the second distance may be determined based on the boundary of the main stream S1 and the sub-stream S2 of the coolant spray stream RSS.

### Implementation of System for Freeze Spraying

To implement the mixing spray system 100 that enables freeze spraying, the coolant spray stream temperature, composition flow rate, composition inflow position described above, or a combination thereof may be considered.

Fundamentally, the mixing spray system 100 needs to be implemented so that the composition is frozen on the skin surface. To achieve this, the temperature of the coolant spray stream RSS formed in the mixing spray system 100 needs to be equal to or less than the freezing point of the composition. In particular, the temperature of the coolant spray stream RSS at a position or region where the composition is introduced needs to be equal to or less than the freezing point of the composition. For example, the freezing point of the composition may be about −30 °C to about 0 °C. In this case, the temperature of the coolant spray stream RSS may be controlled to be equal to or less than about −50 °C or about −30 °C at the position where the composition is introduced.

The mixing spray system 100 needs to be implemented so that the freeze ratio of the composition on the skin surface is equal to or greater than a target freeze ratio. The target freeze ratio refers to a ratio of the solid composition to the composition that reaches the skin surface. Here, the target freeze ratio may be calculated according to the above-described freeze ratio calculation methods.

The target freeze ratio may be determined through freeze spraying experiments. For example, as described later, an effective ratio range in which the penetration effect of the composition is significant may be calculated by conducting an experiment on freeze spraying, and the target freeze ratio may be selected within the effective ratio range. For example, through the first experiment which will be described later, a significant penetration effect was confirmed when the freeze ratio was 17% or 5%, and through the second experiment which will be described later, it was confirmed that the penetration effect increased as the freeze ratio increased, so the target freeze ratio may be determined as equal to or greater than 17%. Alternatively, the target freeze ratio may be determined as equal to or greater than 5%. The target freeze ratio needs to be set as high as possible, but limitations (e.g., prevention of skin damage, prevention of ice film formation, etc.) which will be described later may be further considered.

The composition supply device 2000 may be designed to have an appropriate composition flow rate (e.g., a target flow rate) in consideration of the freeze ratio of the composition. Here, the target flow rate may be a composition flow rate that needs to be sprayed for a predetermined period of time. The pressure that needs to be provided by the actuator 2200 may be specified based on the determined target flow rate. Alternatively, the width of the conduit included in the composition guide 2100 or the width of the conduit connected to the composition guide 2100 may be determined based on the determined target flow rate.

The composition supply device 2000 may be designed to have an appropriate target inflow position for the composition in consideration of the freeze ratio of the composition. That is, the positional relationship between the coolant spray device 1000 and the composition supply device 2000 may be specified to determine the appropriate target inflow position of the composition. As described above, the target inflow position may be determined as a position adjacent to the nozzle 1500 and where vortex formation is minimized based on the orifice of the nozzle 1500. The positional relationship between the nozzle 1500 and the composition guide 2100 of the coolant spray device 1000 may be specified depending on the target inflow position. Specifically, the output end through which the composition is discharged from the composition guide 2100 may be designed to be located at the target inflow position based on the orifice of the nozzle 1500.

The coolant spray device 1000 may be designed so that the coolant has an appropriate temperature in consideration of the freeze ratio of the composition. For example, when spraying the composition and the coolant from the mixing spray system 100, the heat providing unit 1300 may be controlled so that a calculated freeze ratio is equal to or greater than the target freeze ratio. Specifically, when determining the freeze ratio of the composition in the observing region OR of the coolant spray stream RSS while changing the heat energy per unit time produced by the heat providing unit 1300 using the controller 1900, an operating range of the heat providing unit 1300 where the freeze ratio is equal to or greater than the target freeze ratio may be specified.

Here, when the heat providing unit 1300 is implemented as a thermoelectric element, the operating range may refer to a power range that needs to be applied to the heat providing unit 1300. Also, here, when the heat providing unit 1300 is implemented as a thermoelectric element and the controller 1900 applies power to the thermoelectric element through feedback control using a preset target temperature and a real-time temperature of the skin surface, the operating range may refer to a range of the target temperature that needs to be set.

The controller 1900 may control the heat providing unit 1300 so that the heat providing unit 1300 is operated within a specified operating range. For example, the controller 1900 may apply power to the heat providing unit 1300 within a preset power range. The preset power range may refer to a range in which the freeze ratio is 5% to 100%. Alternatively, the preset power range may refer to a range in which the freeze ratio is 17% to 100%. Alternatively, the preset power range may refer to a range in which the freeze ratio is 48% to 100%. Alternatively, the preset power range may refer to a range in which the freeze ratio is 71% to 100%.

Meanwhile, the operating range of the heat providing unit 1300 may be specified by further considering a range in which the temperature of the coolant spray stream RSS does not cause pain or damage to the skin surface. For example, when spraying the composition and the coolant from the mixing spray system 100 onto the skin surface, the heat providing unit 1300 may be determined so that the temperature of the skin surface is equal to or greater than the safe temperature. The safe temperature may be determined within a range of about −10 °C to about 20 °C. For example, the safe temperature may be −10 °C, −9 °C, −8 °C, −7 °C, −6 °C, −5 °C, −4 °C, −3 °C, −2 °C, −1 °C, 0 °C, 1 °C, 2 °C, 3 °C, 4 °C, 5 °C, 6 °C, 7 °C, 8 °C, 9 °C, 10 °C, 11 °C, 12 °C, 13 °C, 14 °C, 15 °C, 16 °C, 17 °C, 18 °C, 19 °C, or 20 °C

Furthermore, the operating range of the heat providing unit 1300 may be specified by further considering a range in which an ice film, which a penetration-impeding substance, is not formed on the skin surface by the coolant spray stream RSS. For example, when the composition and the coolant are sprayed onto the skin surface from the mixing spray system 100, the operating range of the heat providing unit 1300 may be determined so that the temperature of the skin surface is equal to or greater than an ice film forming temperature. Since the ice film may be formed when the skin surface is equal to or less than 0 °C, the ice film forming temperature is 0 °C.

Meanwhile, the controller 1900 may not operate the heat providing unit 1300 or the heat providing unit 1300 may be omitted from the mixing spray system 100. Specifically, as the composition having room temperature is introduced into the coolant spray stream RSS, the temperature of the coolant spray stream RSS may increase. This is because when the temperature of the coolant spray stream RSS is increased above the safe temperature and/or ice film forming temperature by the introduced composition, not heating the coolant before spraying may increase the possibility of freezing or the freeze ratio of the composition.

The coolant pressure may be controlled to control the temperature of the coolant spray stream RSS.

First, the internal pressure of the coolant container RC used in the mixing spray system 100 may be selected appropriately in consideration of the freeze ratio of the composition. For example, the internal pressure of the coolant container RC may be determined within a range of 10 bar to 1,000 bar. Preferably, the internal pressure of the coolant container RC is determined within a range of 30 bar to 200 bar.

The internal pressure of the coolant container RC may be determined in consideration of the target freeze ratio and the safe temperature. For example, the internal pressure of the coolant container RC may be determined between a minimum pressure value at which the freeze ratio of the composition becomes the target freeze ratio and a maximum pressure value at which the temperature of the skin surface becomes the safe temperature.

Meanwhile, the coolant pressure may be controlled by the separate compressor described above and/or a container heater that heats the coolant container RC. The compressor or container heater may control the coolant pressure before spraying to a pressure range determined based on the target freeze ratio and the safe temperature.

In the above, in implementing the mixing spray system 100 for freeze spraying, it has been described that the composition flow rate, the composition inflow position, and the coolant spray stream temperature are sequentially considered. However, the technical idea of the present disclosure is not limited thereto, and the mixing spray system 100 may be designed so that these factors are considered in a different order.

Consequently, the mixing spray system 100 to be implemented includes the coolant container RC that stores the coolant at a pressure of at least 30 bar to 200 bar, the coolant receiving part that receives the coolant from the coolant container RC, a nozzle 1500 having the orifice of a preset size, the composition guide 2100 disposed adjacent to the nozzle 1500 (the output end of the composition guide 2100 is located within a preset distance from the orifice of the nozzle 1500), the flow regulating unit 1200, and the controller 1900. Here, the freeze ratio of the composition in the observing region OR, which is located to be spaced apart from the orifice of the nozzle 1500 by the observing distance OD and has the observing width OW, is equal to or greater than the target freeze ratio. The mixing spray system 100 may further include the distance maintenance unit, and the distance maintenance unit may have a length within the recommended spray distance range to ensure that the freeze ratio of the composition is equal to or greater than the target freeze ratio.

In the above, the mixing spray system 100 implemented to enable freeze spraying has been described. The mixing spray system 100 implemented so that at least a part of the composition is frozen and reaches the skin surface may also be called a freeze spray system.

### Spraying Composition After Freezing

The mixing spray system 100 described above sprays the composition together with the coolant so that the composition is frozen by the coolant during spraying. However, a system that performs freeze spraying cannot be implemented by simply spraying a mixture of the composition and the coolant, and any system may be used for freeze spraying as long as it enables the frozen composition to reach the skin surface. For example, a system that freezes the composition in advance and sprays the frozen composition may be used. Specifically, a system that freezes the composition in advance to generate frozen particles and then sprays the frozen particles onto the skin surface using a transfer medium such as compressed air may be used.

### Freeze Spray Method

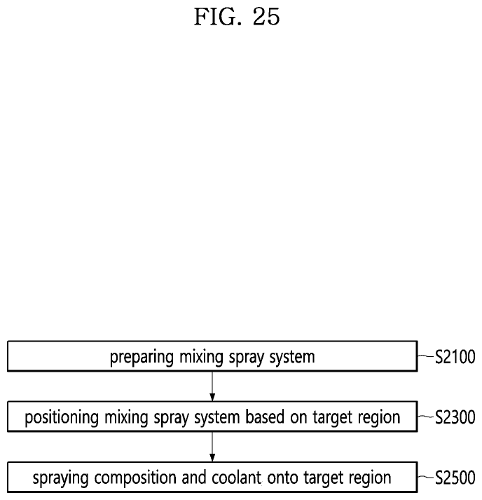

Hereinafter, the freeze spray method using the mixing spray system 100 will be described with reference to FIG. 25.

FIG. 25 is a flowchart illustrating a freeze spray method according to an embodiment.

Referring to FIG. 25, the freeze spray method includes preparing a mixing spray system 100 (S1100), positioning the mixing spray system 100 based on a target region (S1300), and spraying a composition and a coolant together on the target region using the mixing spray system 100 (S1500).

Hereinafter, each step will be described in detail.

First, a user (or operator) may prepare the mixing spray system 100 (S1100). The mixing spray system 100 includes at least a nozzle 1500 for forming a coolant spray stream RSS and a composition guide 2100 disposed adjacent the nozzle 1500. The mixing spray system 100 may include other components described above in addition to the nozzle 1500 and the composition guide 2100.

The user may position the mixing spray system 100 based on the target region (S1300).

For example, the user may position the nozzle 1500 of the mixing spray system 100 at a predetermined distance from the target region.

Here, the predetermined distance may refer to the shortest distance between an orifice of the nozzle 1500 and the target region. The predetermined distance may be substantially the same as the recommended spray distance described above. When the recommended spray distance is presented as a range, the predetermined distance may be included within the recommended spray distance range.

Also, here, a central axis CA of the nozzle 1500 may have a preset angle with respect to the target region. For example, the angle formed between the central axis CA of the nozzle 1500 and a plane including the target region or a virtual plane in contact with the target region may have a value of about 45° to about 90°.

The user may spray the composition and the coolant together on the target region using the mixing spray system 100 (S1500). For example, when the user manipulates an input unit 1700 provided in the mixing spray system 100, a flow regulating unit 1200 may be opened and an actuator 2200 may pressurize the composition, causing the coolant and the composition to be sprayed together on the target region. In a case where the mixing spray system 100 does not include the actuator 2200, when the user manipulates the input unit 1700 (e.g., when the user presses a spray start button), the flow regulating unit 1200 may be opened to form a coolant spray stream RSS, and the composition may be introduced into the coolant spray stream RSS and sprayed due to negative pressure.

When the mixing spray system 100 is operated, the composition stored in a liquid state is introduced into the coolant spray stream RSS. Due to the coolant spray stream RSS, the composition in a liquid state is broken into microparticles, and at the same time, transitions into a solid state in the form of frozen microparticles as the temperature thereof is lowered. The composition in a solid state is accelerated in an acceleration section of the coolant spray stream RSS, and reaches the target region. In addition, when the mixing spray system 100 is viewed from the side at a time point at which the mixing spray system 100 is operated, an observing region OR that is spaced apart by an observing distance OD corresponding to the predetermined distance from the orifice of the nozzle 1500 and has an observing width OW, may be specified, and a freeze ratio in the observing region OR may be 5%.

### Confirmation of Penetration Effect of Freeze Spraying

Hereinafter, a skin penetration effect of freeze spraying will be described through experiments on freeze spraying.

### Experiments Related to Freeze Spraying

The present applicant conducted various experiments related to freeze spraying, and the experiments conducted included an experiment on the penetration effect depending on freezing and an experiment on the penetration effect depending on the freeze ratio.

First, the first experiment that confirmed the penetration effect depending on freezing will be described.

The purpose of the first experiment was to confirm that the penetration effect varies depending on whether a composition is frozen.

In the first experiment, the penetration effect was compared between a case where the composition was sprayed on the skin surface in a non-frozen state (test group 1), a case where the composition was sprayed on the skin surface in a partially frozen state (test group 2 and test group 4), and a case where the composition was sprayed on the skin surface in a mostly frozen state (test group 3).

As a target to which the composition was sprayed, human-derived skin tissue was used. Specifically, facial skin tissue discarded after surgery was used.

In control group, human-derived skin tissue was cut into a predetermined size (2 cm*2 cm) and the composition was applied thereon. The composition included acetyl hexapeptide-8-FITC combined with a fluorescent substance (fluorescein isothiocyanate, FITC) to confirm the penetration effect.

Then, after 24 hours, a fluorescence image of the cross-section of the human-derived skin tissue was captured, and the intensity and penetration depth of the fluorescent substance located under the epidermis were measured.

In test group 1, human-derived skin tissue was cut to a predetermined size and the composition was sprayed thereon without freezing. The composition included antifreeze (PG) to prevent freezing by a coolant, and acetyl hexapeptide-8-FITC combined with a fluorescent substance (FITC) to confirm the penetration effect.

The above-described mixing spray system 100 was used to spray the composition. Specifically, the coolant spray device 1000 described above and the composition supply device 2000 according to the second embodiment were used. For the mixing spray system 100 used, the freeze ratio was calculated to be 0%.

Then, after 24 hours, a fluorescence image of the cross-section of the human-derived skin tissue was captured, and the intensity and penetration depth of the fluorescent substance located under the epidermis were measured.

In test group 2, human-derived skin tissue was cut to a predetermined size, and only a part of the composition was frozen and sprayed thereon. The composition included acetyl hexapeptide-8-FITC combined with a fluorescent substance (FITC) to confirm the penetration effect.

The above-described mixing spray system 100 was used to spray the composition. Specifically, the coolant spray device 1000 described above and the composition supply device 2000 according to the second embodiment were used. For the mixing spray system 100 used, the freeze ratio was calculated to be 17% (within a measurement error of 5%, about 12% to about 22%).

Then, after 24 hours, a fluorescence image of the cross-section of the human-derived skin tissue was captured, and the intensity and penetration depth of the fluorescent substance located under the epidermis were measured.

In test group 3, human-derived skin tissue was cut to a predetermined size, and most of the composition was frozen and sprayed thereon. The composition included acetyl hexapeptide-8-FITC combined with a fluorescent substance (FITC) to confirm the penetration effect.

The above-described mixing spray system 100 was used to spray the composition. Specifically, the coolant spray device 1000 described above and the composition supply device 2000 according to the second embodiment were used. For the mixing spray system 100 used, the freeze ratio was calculated to be 100% (within a measurement error of 1%, about 99% to about 100%).

Then, after 24 hours, a fluorescence image of the cross-section of the human-derived skin tissue was captured, and the intensity and penetration depth of the fluorescent substance located under the epidermis were measured.

In test group 4, human-derived skin tissue was cut to a predetermined size, and only a part of the composition was frozen and sprayed thereon. The composition included acetyl hexapeptide-8-FITC combined with a fluorescent substance (FITC) to confirm the penetration effect.

The above-described mixing spray system 100 was used to spray the composition. Specifically, the coolant spray device 1000 and the composition supply device 2000 according to the first embodiment were used. For the mixing spray system 100 used, the freeze ratio was calculated to be 5% (within a measurement error of 1%, about 4% to about 6%).

Then, after 24 hours, a fluorescence image of the cross-section of the human-derived skin tissue was captured, and the intensity and penetration depth of the fluorescent substance located under the epidermis were measured.

The present applicant conducted the second experiment to confirm the penetration effect depending on the freeze ratio.

The purpose of the second experiment was to confirm that the penetration effect varies depending on the freeze ratio.

In the second experiment, a composition was sprayed on the skin surface in a frozen state, and the penetration effect was compared between a case where the freeze ratio of the composition was 5% (within a measurement error of 1%, about 4% to about 6%) (test group 1), a case where the freeze ratio of the composition was 17% (within a measurement error of 5%, about 12% to about 22%) (test group 2), a case where the freeze ratio of the composition was 48% (within a measurement error of 3%, about 45% to about 51%) (test group 3), a case where the freeze ratio of the composition was 71% (within a measurement error of 3%, about 68% to about 74%) (test group 4), and a case where the freeze ratio of the composition was 100% (within a measurement error of 1%, about 99% to about 100%) (test group 5).

The mixing spray system 100 according to the second embodiment was used to spray the composition.

As a method of adjusting the freeze ratio, a method of adjusting the heat energy applied to a coolant before spraying from the heat providing unit 300 of the mixing spray system 100 was adopted.

More specifically, the heat providing unit 1300 included a thermoelectric element and the power applied to the thermoelectric element was adjusted. As the heat energy applied before the coolant was sprayed through the nozzle 1500 changed, the temperature of a coolant spray stream RSS formed in the nozzle 1500 changed, and the freeze ratio of the composition changed accordingly.

Here, the applied power was adjusted using a pulse width modulation (PWM) method, and the heating power applied using the PWM method was divided into levels from 0 to 999. From test group 1 to test group 5, the freeze ratio was adjusted by varying the heating power applied to the thermoelectric element.

In the second experiment, hydrogel was used as a target to which the composition and the coolant were sprayed, and the penetration effect was compared by the number of holes formed in the hydrogel. The number of holes formed in the hydrogel was determined with the naked eye, and it was determined that as the number of holes formed after the composition and the coolant were sprayed on the hydrogel for a predetermined period of time increased, the penetration effect increased.

### Experimental Results and Analysis

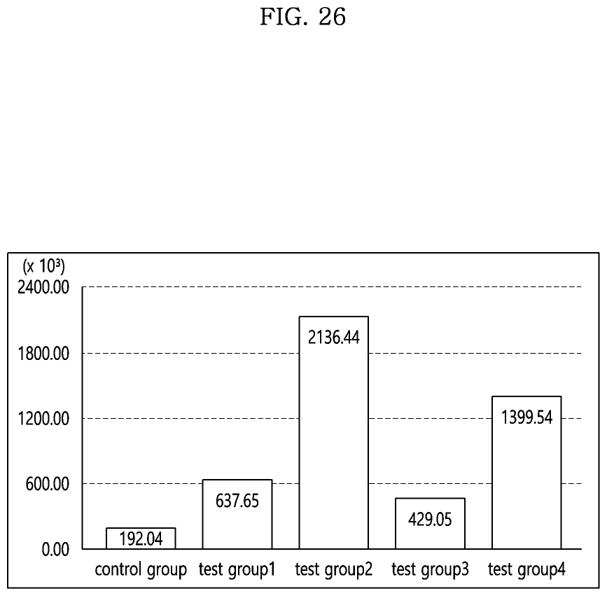

Hereinafter, the results of the first and second experiments will be described with reference to FIGS. 26 to 29.

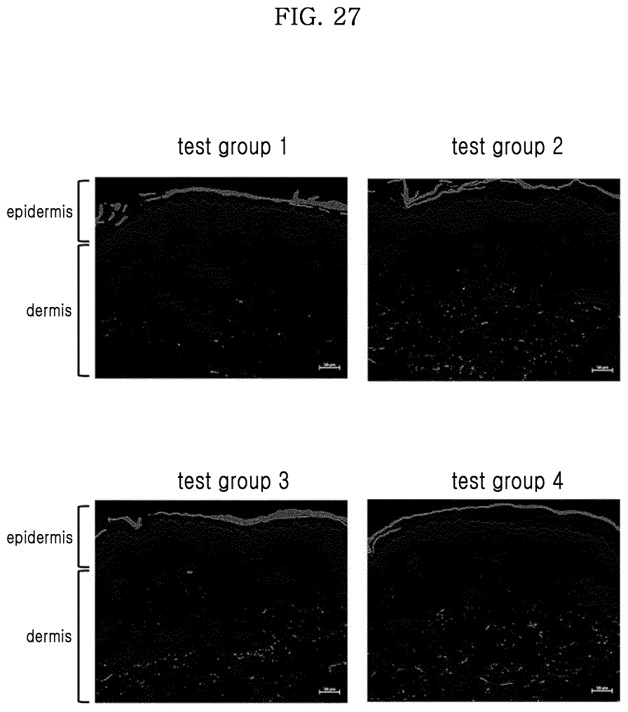

FIGS. 26 and 27 are views illustrating the results of the first experiment to confirm the penetration effect depending on freezing and the penetration effect depending on the freeze ratio.

According to the results of the first experiment, the penetration effect when the composition was sprayed in a frozen state (test group 2 and test group 4) was significantly higher than the penetration effect when the composition was sprayed in a non-frozen state (test group 1).

Referring to FIG. 26, the fluorescence intensity was 192.04 in control group and 637.65 in test group 1, while the fluorescence intensity was 2136.44 in test group 2 and 1399.54 in test group 4. Compared to test group 1, it is understood that the penetration effect improved by about 3.35 times in test group 2, and by about 2.19 times in test group 4.

Furthermore, when observing a fluorescence image of the cross-section of skin tissue in each case, as illustrated in FIG. 27, it was confirmed that the amount of composition penetrated was significantly increased in test group 2 and test group 4 compared to test group 1.

Meanwhile, in test group 3, the fluorescence intensity was measured at 429.05, which was lower than that of test group 1. This is because, as described above, the low temperature of the sprayed coolant caused an ice film to be formed on the surface of human-derived skin tissue, and as a result, a large amount of frozen particles did not reach the skin tissue surface. It is expected that the ice film will not be formed in areas where body heat exists, such as human skin, and therefore, the penetration effect is expected to be high when the conditions of test group 3 are applied to human skin.

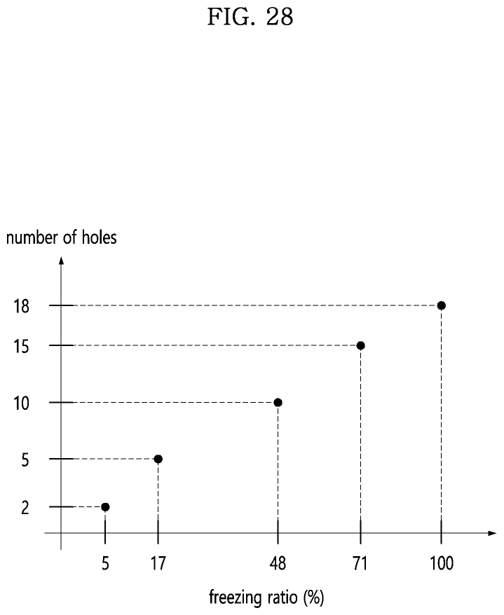

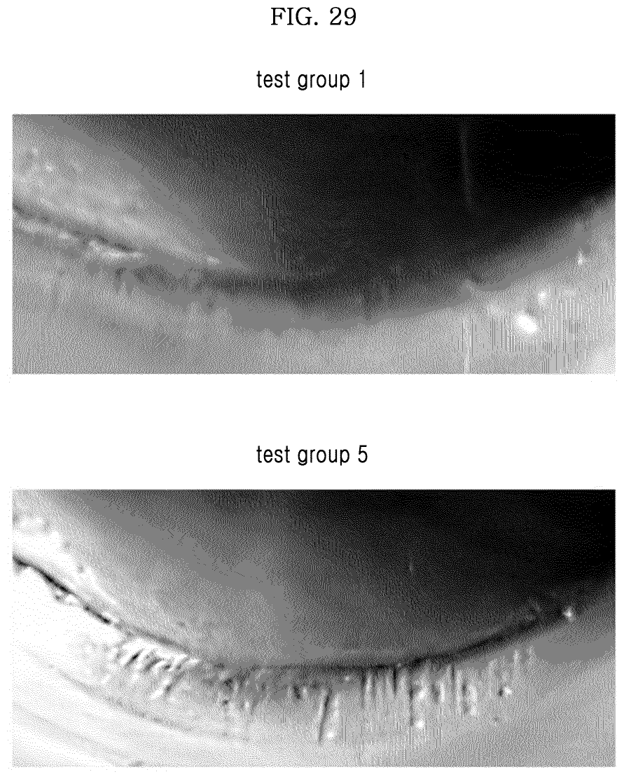

FIG. 28 is a view illustrating the results of the second experiment to confirm the penetration effect depending on the freeze ratio. FIG. 29 is a view illustrating the penetration effect of test group 1 and test group 5 in the second experiment

As a result of the second experiment, it was confirmed that the penetration effect improved as the freeze ratio increased.

Referring to FIG. 28, the number of holes was 2 when the freeze ratio was 5%, the number of holes was 5 when the freeze ratio was 17%, the number of holes was 10 when the freeze ratio was 48%, the number of holes was 15 when the freeze ratio was 71%, and the number of holes was 18 when the freeze ratio was 100%. This thus confirmed that the number of holes increased as the freeze ratio increased. As a result, it can be understood that the penetration effect improves as the freeze ratio increases.

The penetration effect can also be confirmed with the naked eye. As illustrated in FIG. 29, the number of holes formed in the hydrogel in test group 5 was significantly increased compared to test group 1.

According to the first experiment, the penetration effect of the composition was significant when the freeze ratio of the composition was 5% and 17%. In addition, according to the second experiment, the penetration effect improved when the freeze ratio of the composition was 17% compared to when the freeze ratio was 5%, the penetration effect improved when the freeze ratio of the composition was 48% compared to when the freeze ratio was 17%, the penetration effect improved when the freeze ratio of the composition was 71% compared to when the freeze ratio was 48%, and the penetration effect improved when the freeze ratio of the composition was 100% compared to when the freeze ratio was 71%.

Therefore, the effective ratio range may be 5% to 100%. Alternatively, the effective ratio range may be 5% to 17%. Alternatively, the effective ratio range may be 5% to 48%. Alternatively, the effective ratio range may be 5% to 71%. Alternatively, the effective ratio range may be 17% to 48%. Alternatively, the effective ratio range may be 17% to 71%. Alternatively, the effective ratio range may be 17% to 100%. Alternatively, the effective ratio range may be 48% to 71%. Alternatively, the effective ratio range may be 48% to 100%. Alternatively, the effective ratio range may be 71% to 100%.

Considering the measurement error range, the effective ratio range may be 2% to 100%. Alternatively, the effective ratio range may be 2% to 22%. Alternatively, the effective ratio range may be 2% to 51%. Alternatively, the effective ratio range may be 2% to 74%. Alternatively, the effective ratio range may be 12% to 51%. Alternatively, the effective ratio range may be 12% to 74%. Alternatively, the effective ratio range may be 12% to 100%. Alternatively, the effective ratio range may be 45% to 74%. Alternatively, the effective ratio range may be 45% to 100%. Alternatively, the effective ratio range may be 68% to 100%.

The mixing spray system 100 may be designed so that the freeze ratio of the sprayed composition is within the effective ratio range.

### Control of Freeze Spraying Characteristics

In the above, the meaning of freeze spraying, the mixing spray system 100 for performing the freeze spray method, and the confirmation of the penetration effect through freeze spraying-related experiments has been described.

In freeze spraying, frozen particles of the composition may have the following characteristics: particle size and spray speed. These characteristics may be important control factors in commercializing the mixing spray system 100 that performs freeze spraying.

For a composition having a specific viscosity and surface tension, the size of frozen particles of the composition may be controlled by the temperature of the coolant spray stream RSS, the flow rate of the coolant spray stream RSS, and the composition flow rate. In addition, the size of the frozen particles may be controlled by the shape of the output end of the composition guide 2100 where the composition is located immediately before the composition is introduced into the coolant spray stream RSS. For example, the output end of the composition guide 2100 may have a blunt or pointed shape, and the size of the frozen particles of the composition may be larger in the blunt shape than in the pointed shape.

### Control of Frozen Particle Size in Freeze Spraying

Frozen particles formed by freezing the liquid composition have a particle size. As the size of the frozen particles of the composition decreases, the mass of the coolant in the coolant spray stream RSS increases relative to the mass of the composition that has an initial velocity of substantially zero immediately after the composition is introduced into the coolant spray stream RSS. This means that according to the law of conservation of momentum, as the size of the frozen particles of the composition decreases, the velocity of the frozen particles of the composition increases. However, when the size of the frozen particles of the composition excessively decreases, an increase in the surface area relative to the momentum may increase air resistance. As a result, deceleration of the spray speed may increase while the solid composition reaches the skin surface.

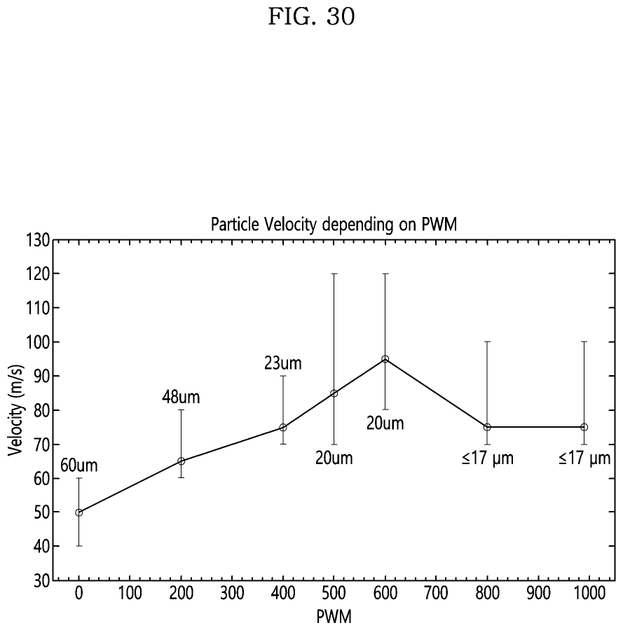

FIG. 30 is a view illustrating the relationship between particle size and spray speed according to an embodiment. The frozen particle size indicated at each point of the graph in FIG. 30 refers to the average particle size of at least a part of frozen particles of the composition. In obtaining the graph illustrated in FIG. 30, the mixing spray system 100 according to the second embodiment was used, and PWM control was used as a method of controlling the frozen particle size of the composition. Specifically, as the level of heating power applied through the PWM method increased, the frozen particle size of the composition decreased.

Referring to FIG. 30, it can be seen that the speed of the frozen particles of the composition increased as the particle size of the composition decreased, but when the particle size of the composition decreased to equal to or less than a predetermined level, the velocity of the frozen particles of the composition decreased.

Therefore, the size of the frozen particles of the composition may be set based on the point at which the velocity of the frozen particles of the composition is maximum. For example, the mixing spray system 100 may be designed so that the average size of the frozen particles of the composition is equal to or less than about 60 μm or is close to about 20 μm. For another example, when considering the direction of increasing the freeze ratio of the composition, the mixing spray system 100 may be designed so that the average size of the frozen particles of the composition is equal to or less than about 150 μm or is close to about 70 μm. As another example, the mixing spray system 100 may be designed so that the average size of the frozen particles of the composition is within about 20 μm to about 60 μm, about 20 μm to about 100 μm, about 10 μm to 60 μm, or about 10 μm to 300 μm.

In the above, the average size of the frozen particles refers to the average size of the composition particles frozen in the coolant spray stream RSS. However, the above-described method of calculating the average size of the frozen particles of the composition in the observing region OR may be used as a method of calculating the average size of the frozen particles of the composition.

Here, the above-described method of controlling the temperature of the coolant spray stream RSS may be used as a method of controlling the frozen particle size. For example, the frozen particle size of the composition may be controlled by controlling the degree to which the coolant is heated by the heat providing unit 1300 before being sprayed.

Meanwhile, the particle size of the frozen composition (or solid composition) excessively increases, the frozen particles may stimulate pain points when sprayed on the skin surface, causing pain. Specifically, pain may be caused when the size of the frozen particles is excessively larger than the size of skin cells (about 30 μm) or the size of pores on the skin surface (about 20 μm to about 50 μm). Therefore, the mixing spray system 100 may be designed so that the size of the frozen particles is less than about 20 μm to about 50 μm.

The size of the frozen particles of the composition may be controlled to control the skin penetration depth of the composition.

The frozen particles melt due to body heat after penetrating into the epidermis. Since the melting time varies depending on the size of the frozen particles, the position where the composition melts within the skin varies. By controlling the size of the frozen particles, the position where the composition melts may be controlled, and the depth where the composition penetrates may be controlled accordingly.

For example, at the same velocity condition, as the size of the frozen particles increases, the penetration depth may increase, and as the size of the frozen particles decreases, the penetration depth may decrease. Therefore, after determining the target penetration depth and specifying the frozen particle size range corresponding to the determined penetration depth, at least one of the level of heating power applied to the heat providing unit 1300 based on the corresponding frozen particle size range, the temperature of the coolant container RC, the shape of the output end of the composition guide 2100, the adhesive strength between the composition guide 2100 and the composition, and the flow rate of the composition may be selectively determined.

In addition, for a composition that is not appropriate for penetrating into the dermis of the skin, for example, a composition that is not classified as a medicine or drug, the size of frozen particles of the composition may be made small in order to limit the penetration depth to the stratum corneum. For example, the mixing spray system 100 may be designed/controlled so that the size of the frozen particles of the composition is about 10 μm to about 50 μm.

### Control of Spray Speed

The penetration effect may be improved as the velocity of frozen particles when the composition reaches the skin surface increases.

As a method of increasing the speed of the frozen particles of the composition, there is a method of reducing the size of the frozen particles. For example, the amount of coolant heated before spraying may be increased to reduce the size of the frozen particles. Alternatively, the shape of the end of the composition guide 2100 may be manufactured to be more sharp (pointed).

As a method of increasing the speed of the frozen particles of the composition, there is a method of increasing the spray speed of coolant particles that transfer kinetic energy to the composition. For example, the spray speed of the coolant particles may be increased by increasing the internal pressure of the coolant container RC in which the coolant is stored.

In order to increase the speed of the frozen particles of the composition, the length of the above-described acceleration section may be secured at equal to or greater than a predetermined level. In other words, the coolant and the composition may be sprayed while the skin surface and the nozzle 1500 are spaced apart from each other by equal to or greater than a minimum acceleration distance. Here, the minimum acceleration distance may be selected within a range of about 3 mm to about 5 mm or about 1 mm to about 10 mm.

The mixing spray system 100 may be designed so that the speed of the frozen particles of the composition is equal to or greater than 50 m/s. Alternatively, the mixing spray system 100 may be designed so that the speed of the frozen particles of the composition is equal to or greater than 60 m/s, 70 m/s, 80 m/s, 90 m/s, 100 m/s, 110 m/s, or 120 m/s. Here, the speed of the frozen particles of the composition may refer to the average moving speed of composition particles moved in the coolant spray stream RSS. For example, the speed of the frozen particles of the composition was obtained by tracking each frozen particle in an image captured with the high-speed camera, measuring the moving speed, and calculating the average value. Here, the speed of the frozen particles may be calculated as the average speed of at least parts of particles in the coolant spray stream RSS. Alternatively, the speed of the frozen particles may be calculated as the average speed of particles in the above-described observing region OR.

Meanwhile, by increasing the spray speed of the composition, the composition may be prevented from decelerating due to air resistance until it reaches the skin surface. For example, formation of a sheath jet around the coolant spray stream RSS may prevent the composition particles from encountering air. To form the sheath jet, the mixing spray system 100 may further include a sheath nozzle. The sheath nozzle may spray the coolant or compressed air to surround the coolant spray stream RSS. The sheath nozzle is disposed around the nozzle 1500 and may have a ring-shaped spray port. Alternatively, the sheath nozzle may be composed of a plurality of nozzles arranged around the nozzle 1500. When the temperature of the sheath jet is equal to or less than a predetermined level (e.g., the freezing point of the composition), the temperature of the frozen particles of the composition may be prevented from lowering.

### Post-Needling Freeze Spray Method

The freeze spray method described above may be used in conjunction with the needling method to perform a post-needling freeze spray method. For example, the post-needling freeze spray method may include the following steps: preparing a needling device 3000 and a mixing spray system 100; forming a plurality of holes by contacting a needling structure 3100 of the needling device 3000 with a target region; and freezing and spraying a composition onto the target region.

The needling device 3000 is a device including a needle configured to form a hole, as described above, and may be of a roller type, a stamp type, or a punching (automatic needling) type for use.

The mixing spray system 100 refers to a device configured to spray the coolant and the composition together. However, the mixing spray system 100 can be implemented to freeze and spray the composition. For example, a coolant spraying device 1000 may be implemented such that the freeze ratio of the composition in an observing region OR equals or exceeds a preset value (for example, 5%, 17%, 48%, or 71%). Specifically, the coolant spraying device 1000 includes a heat providing unit 1300. During operation of the coolant spraying device 1000, power within a power range in which the freeze ratio becomes equals or exceeds the preset value may be applied to the heat providing unit 1300.

In addition, the composition supply device 2000, according to the first or second embodiment, may be used.

In the meantime, during the post-needling freeze spray method, the frozen particle size may be controlled. For example, the target frozen particle size of the composition to be sprayed from the mixing spray system 100 may be determined on the basis of the needle width NW of the needling device 3000. Specifically, the target size may be determined to be smaller than the needle width NW.

### Needling and Mixing Spray System Enabling Post-Needling Mixing Spraying

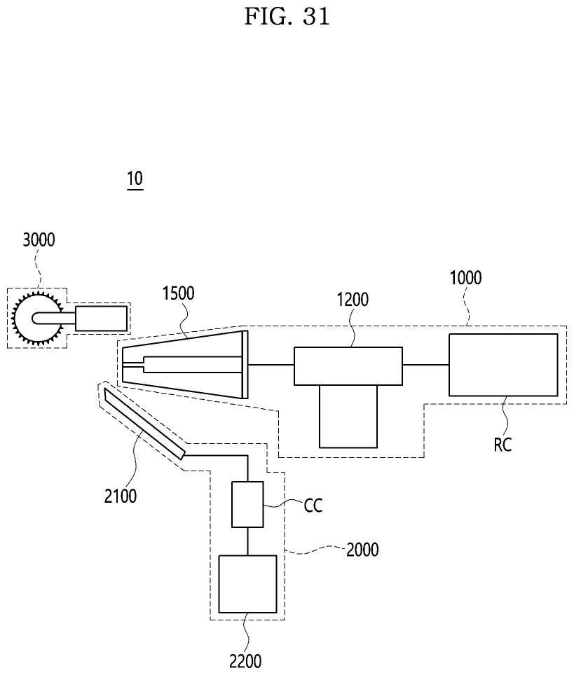

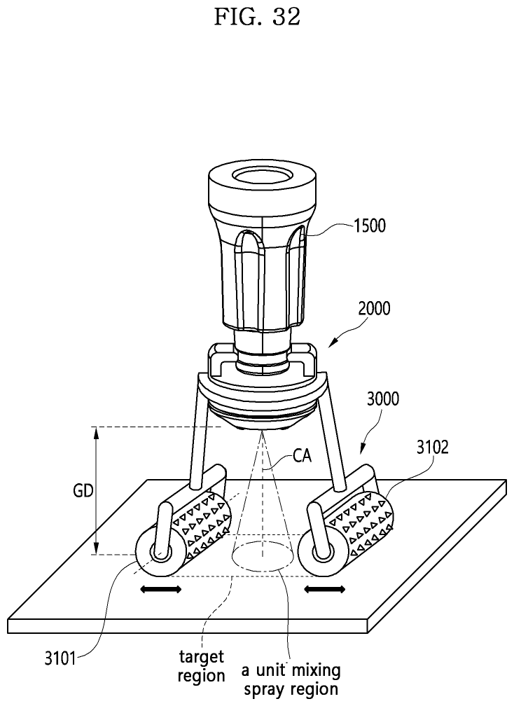

Hereinafter, a needling and mixing spray system enabling post-needling mixing spraying is to be described with reference to FIGS. 31 and 32.

FIG. 31 is a diagram illustrating the needling and mixing spray system 10 according to an embodiment.

FIG. 32 is a diagram illustrating a form in which the needling and mixing spray system 10 is implemented according to an embodiment, and its use.

Referring to FIG. 31, the needling and mixing spray system 10 may include a coolant spraying device 1000, a composition supply device 2000, and a needling device 3000. The needling and mixing spray system 10 may be understood as an integrated device capable of performing a coolant spray function, a composition supply function, and a needling function.

A user may perform the needling method on a target region using the needling and mixing spray system 10 and may spray a composition and a coolant onto the target region using the needling and mixing spray system 10.

The coolant spraying device 1000, the composition supply device 2000, and the needling device 3000, included in the needling and mixing spray system 10, may be applied in the same manner as described above. However, the respective devices may be assembled or combined with one another. To this end, some components included in the devices may be omitted, or additional components may be provided.

### Mounting Method of Needling Device

In one example, the needling device 3000 may be coupled to the coolant spraying device 1000.

Specifically, the connecting frame 3200 of the needling device 3000 may be configured to be connected to the nozzle 1500 of the coolant spraying device 1000. The connecting frame 3200 may, for example, be coupled to the housing or nozzle 1500 of the coolant spraying device 1000 through screw coupling, hook coupling, force-fitting coupling, or the like. The needling device 3000 may not include the grip member 3300.

Alternatively, the needling device 3000 further includes a device coupling member, and the connecting frame 3200 may be connected to the coolant spraying device 1000 through this device coupling member. The device coupling member may be configured to be coupled to the housing of the coolant spraying device 1000. In this case, the device coupling member may have a shape that surrounds the nozzle 1500, replacing the cover COV of the coolant spraying device 1000. The needling device 3000 may not include the grip member 3300.

When the needling device 3000 is coupled to the coolant spraying device 1000, the positional relationship between the grip part in the housing of the coolant spraying device 1000 and the needling structure 3100 may be considered. In this case, the grip part refers to a part of the coolant spraying device 1000 that is gripped by the user and, for example, has a cylindrical or polygonal column shape. When the needling structure 3100 is of a roller type, the needling device 3000 may be coupled to the coolant spraying device 1000 so that the rolling direction of the needling structure 3100 is parallel to the central axis of the grip part.

The needling device 3000 is attachable to and detachable from the coolant spraying device 1000. This is because, when the needling device 3000 used is disposable or requires disinfection, this needling device 3000 must be detachable from the coolant spraying device 1000 and replaceable.

In another example, the needling device 3000 may be coupled to the composition supply device 2000.

Specifically, the connecting frame 3200 of the needling device 3000 may be configured to be connected to the composition guide 2100 or the mixing unit 2300 of the composition supply device 2000. The connecting frame 3200 may, for example, be coupled to the housing, the composition guide 2100, or the mixing unit 2300 of the composition supply device 2000 through screw coupling, hook coupling, force-fitting coupling, or the like. The needling device 3000 may not include the grip member 3300.

Alternatively, the needling device 3000 further includes a device coupling member, and the connecting frame 3200 may be connected to the composition supply device 2000 through this device coupling member. The device coupling member may be configured to be coupled to the housing, the composition guide 2100, or the mixing unit 2300 of the composition supply device 2000. The needling device 3000 may not include the grip member 3300.

The needling device 3000 is attachable to and detachable from the composition supply device 2000. This is because, when the needling device 3000 used is disposable or requires disinfection, this needling device 3000 must be detachable from the composition supply device 2000 and replaceable.

### Plurality of Needling Structures

The needling and mixing spray system 10 may include a plurality of needling structures 3100. Referring to FIG. 32, the needle device 3000 includes a first needling structure 3101 and a second needling structure 3102, and the first needling structure 3101 and the second needling structure 3102 may be disposed such that the central axis of the nozzle 1500 is positioned therebetween.

The first needling structure 3101 and the second needling structure 3102 may be implemented as a roller type, and the central axis of the first needling structure 3101 may be practically parallel to the central axis of the second needling structure 3102.

The needling and mixing spray system 10 includes the first needling structure 3101 and the second needling structure 3102. As a result, when performing the needling method and the mixing spray method on the target region, the needling method may be performed on the target region before the mixing spray method. More specifically, when the user sprays the coolant and the composition while moving the needling and mixing spray system 10 on the target region, holes are formed in the target region by the first needling structure 3101 while moving the needling and mixing spray system 10 in a direction toward the first needling structure 3101 from the second needling structure 3102. Then, the coolant and the composition are sprayed. On the contrary, when the needling and mixing spray system 10 moves in a direction toward the second needling structure 3102 from the first needling structure 3101, holes are formed in the target region by the second needling structure 3102, followed by spraying the coolant and the composition.

This can prevent a problem in which, because the direction of performing the needling method varies depending on the user, mixing spray is performed before the needling according to the performing direction when a single needling structure 3100 is used. In other words, as the needling structure 3100 is disposed in both directions around the central axis CA of the nozzle 1500, the order of performing the needling method and mixing spraying on the target may be fixed as needling followed by mixing spraying, regardless of the direction in which the user moves the needling and mixing spray system 10.

### Positional Relationship Between Needling Device and Nozzle

In the needling and mixing spray system 10, the needling device 3000 and the nozzle 1500 must be disposed such that specific conditions are to be satisfied.

Specifically, when the user sequentially performs the needling method and the mixing spray method using the needling and mixing spray system 10, the needling structure 3100 is positioned on the target region to form holes, and the composition and the coolant may be sprayed, with the nozzle 1500 disposed while being directed toward the target region.

In this case, the needling device 3000 and the nozzle 1500 need to be disposed such that a unit mixing spray region where the coolant and the composition are to be sprayed does not meet the needling structure 3100. For example, The distance from the central axis CA of the nozzle 1500 to the needling structure 3100, has a greater value than the radius of the unit mixing spray region.

### Distance Maintenance Function of Needling Device

In spraying the composition and the coolant onto the target region, the spray distance between the target region and the nozzle 1500 of the coolant spraying device 1000 is preferably maintained within a recommended spray distance range.

In the needling and mixing spray system 10, the needling device 3000 may be directed such that the spray distance is maintained within the recommended spray distance range. Specifically, while the needling device 3000 is in contact with the target region, the needling and mixing spray system 10 is to be used such that the composition and the coolant are sprayed. Thus, as long as the distance from the nozzle 1500 to the end of the needling device 3000 is maintained within the recommended spray distance range, the spray distance from the nozzle 1500 to the target region can also be maintained within the recommended spray distance range.

More specifically, referring to FIG. 32, the end of the needling structure 3100 may be spaced apart from the orifice of the nozzle 1500 by a guide distance GD in a direction parallel to the central axis CA of the nozzle 1500, and the guide distance GD may fall within the recommended spray distance range.

In the meantime, when the needling structure 3100 of the needling device 3000 is a roller, at least a portion of the part in the needling device 3000, the part contacting with the skin surface, may be implemented in a “U-shape”. This is because the angle of the coolant spraying device 1000 with respect to the skin surface, when the needling structure 3100 is a roller, may be changed while the needling and mixing spray system 10 is in contact with the skin surface, in other words, meaning that the region toward which the nozzle 1500 is directed and the distance between the nozzle 1500 and the skin surface are not fixed.

### Device Control Method Based on Usage Scenario

The needling and mixing spray system 10 has various uses. Hereinafter, the use of the needling and mixing spray system 10 and the corresponding control method are to be described.

The user may simultaneously spray the composition and the coolant while performing needling on the target region using the needling and mixing spray system 10. Specifically, the user may bring the needling structure 3100 into contact with the target region, followed by moving the needling structure 3100 (for example, by rolling or rubbing) while simultaneously operating the input unit 1700 of the coolant spraying device 1000 (for example, by pressing the spray start button), thereby spraying the composition and the coolant. In this case, the needling method and the mixing spray method may be understood to be initiated by the user.

The user may perform needling on the target region using the needling and mixing spray system 10, but the composition and the coolant may be automatically sprayed. Specifically, when the user brings the needling structure 3100 into contact with the target region and then moves the needling structure 3100 (for example, by rolling or rubbing), the coolant spraying device 1000 detects this movement, so the flow regulating unit 1200 may be opened. The needling and mixing spray system 10 may further include a needling sensor configured to detect the rotation, movement, and contact of the needling structure 3100 with the target region. The controller 1900 of the needling and mixing spray system 10 may open the flow regulating unit 1200 when receiving a signal from the needling sensor or may operate the actuator 2200 while opening the flow regulating unit 1200.

When the needling device 3000 is implemented as a punching type, the controller 1900 of the needling and mixing spray system 10 may control the punching operation of the needling structure 3100 and the opening and closing operation of the flow regulating unit 1200.

In one example, the needling and mixing spray system 10 may be operated, with the needling device 3000 being in contact with a first region of the target region while positioning the nozzle 1500 to be directed toward a second region of the target region. In this case, holes may be formed in the first region, and the composition and the coolant may be sprayed onto the second region. Thereafter, the needling and mixing spray system 10 may be operated, with the needling device 3000 being in contact with a third region of the target region while positioning the nozzle 1500 to be directed toward the first region. In this case, the composition and the coolant may be sprayed onto the first region where the holes have been formed, and holes may be formed in the third region. As described, the needling and mixing spray system 10 can simultaneously perform the needling method on one region while performing the mixing spray method on another region. When the user properly moves the needling and mixing spray system 10, the composition and the coolant may be sprayed onto the region where holes have been formed, thereby forming holes in a new region.

In this case, when receiving an operation start input through the input unit 1700, the controller 1900 may control the needling device 3000 so that the needling structure 3100 performs a punching operation for a preset time, and may control the flow regulating unit 1200 to be opened for a preset time.

Furthermore, the controller 1900 may perform a control method of reciprocating the needling structure 3100 for a preset time and opening the flow regulating unit 1200 at a preset cycle. The controller 1900 may, for example, perform the control method described above at intervals of 0.5 to 5 seconds, and the user may move the needling and mixing spray system 10 at each cycle. In the meantime, the controller 1900 may also continuously perform the control method described above.

### Alternative Needling Method

Hereinabove, the post-needling mixing spray method has been described, where the needling method has aimed to form a channel through which the composition could reach a depth beneath the epidermis of the skin. More specifically, it is extremely difficult for the composition to pass through the stratum corneum of the epidermis when penetrating the skin. For this reason, it can be understood that the needling method has been employed as one way of enabling the composition to pass through the stratum corneum.

Therefore, any method of weakening or partially removing the stratum corneum that significantly interferes with the penetration of the composition can replace the needling method.

In one example, a laser irradiation method, instead of the needling method, may be used. Specifically, when the skin is irradiated using a laser (for example, a CO2 laser, a radio-frequency (RF) laser, or the like) having a micro-scale spot size and an intensity within a predetermined range, micro-holes can be formed in the skin surface. Therefore, when spraying the composition and the coolant after irradiating the target region using a laser, the penetration effect can be improved by spraying the composition through the micro-holes formed by the laser.

In another example, a micro-plasma generation method, instead of the needling method, may be used. Specifically, micro-holes can be formed in the skin surface when high-frequency energy is applied to an electrode having a micro-scale width while the electrode is in contact with the skin surface. Therefore, when spraying the composition and the coolant after generating micro-plasma in the target region, the penetration effect can be improved by spraying the composition through the micro-holes formed by the plasma.

In a further example, a chemical peeling method, instead of the needling method, may be used. Specifically, when applying a drug (for example, trichloroacetic acid (TCA), phenol, alpha-hydroxy acid (AHA), beta-hydroxy acid (BHA), Jessner's Solution, or the like) onto the skin surface, the stratum corneum of the skin may be removed. Therefore, when applying a drug for removing the stratum corneum onto the target region and spraying the composition and the coolant, the penetration effect can be improved by spraying the composition with the stratum corneum removed.

In yet another example, an RF needling method, instead of the needling method, may be used. Specifically, the RF needling method induces the effect of promoting skin regeneration and collagen production by applying high-frequency current with microneedles inserted into the skin. Even in the RF needling method, microneedles are used to form holes in the skin surface. Thus, when spraying the composition and the coolant after RF needling, the penetration effect of the composition can be improved.

The features, structures, effects, etc. described in the embodiments are included in at least one embodiment of the present disclosure and are not necessarily limited to one embodiment. Furthermore, the features, structures, effects, etc. provided in each embodiment can be combined or modified in other embodiments by those skilled in the art to which the embodiments belong. Therefore, the contents related to the combination and modification should be construed to be included in the scope of the present disclosure.

The embodiments described herein have been provided merely for illustrative purposes, those skilled in the art will appreciate that various modifications, additions, and substitutions are possible, without departing from the scope and spirit of the present disclosure. In other words, each element specifically shown in the embodiments can be implemented in modified forms. Therefore, the contents related to the combination and modification should be construed to be included in the scope of the present disclosure as disclosed in the accompanying claims.

What is claimed is:

1. A method for penetrating a composition into a depth beneath an epidermis of a target region which is a surface of a skin, the method comprising:
contacting a plurality of microneedles to the target region,
wherein each of the plurality of microneedles has a needle width with 0.3 mm or smaller,
wherein the contact between the plurality of microneedles and the target region forms a plurality of holes in the target region; and
spraying, to the target region in which the plurality of holes are formed, a composition including an active ingredient in a liquid or emulsion type together with a coolant, such that a temperature of the target region is cooled by the coolant to 10 degrees Celsius or below while the composition collides with the target region and penetrates below the epidermis of the target region.

2. The method of claim 1, wherein spraying the composition is performed within a pre-configured time after the contacting the plurality of microneedles to the target region.

3. The method of claim 2, wherein the pre-configured time is 10 seconds or less.

4. The method of claim 1, wherein contacting the plurality of microneedles to the target region comprises:
dividing the target region into at least a first region and a second region;
sequentially contacting the plurality of microneedles to the first region and the second region,
wherein the composition and the coolant are not sprayed to the first region during the plurality of microneedles is contacted to the second region.

5. The method of claim 1, wherein a needling device including a roller having the plurality of microneedles protruding from an outer surface of the roller is used in the contacting the plurality of microneedles to the target region, and
wherein the plurality of microneedles contact the target region while the roller is rolled on the target region thereby forming the plurality of holes.

6. The method of claim 1, wherein the needle length is 0.1 mm or longer and 3.0 mm or shorter.

7. The method of claim 1, wherein the needle length is 1.5 mm.

8. The method of claim 1, wherein the needle length is 0.5 mm.

9. The method of claim 1, wherein a mixing spray system is used in the spraying the composition with the coolant to the target region, and
wherein the mixing spray system comprises a coolant spray device including a nozzle for spraying the coolant and a composition supply device configured to introduce a composition into a coolant spray stream formed by the nozzle.

10. The method of claim 1, wherein a portion of the composition sprayed to the target region is frozen by a coolant and reaches to the target region in a solid state.

11. The method of claim 10, wherein a freezing rate of the composition reached to the target region is 5% or more.

12. A needling and mixing spray system, comprising:
a coolant container configured to accommodate a coolant;
a coolant receiving unit configured to receive the coolant from the coolant container;
a valve fluidically connected to the coolant receiving unit, wherein the coolant output from the coolant receiving unit flows into the valve;
a nozzle fluidically connected to the valve and having an orifice of a pre-configured diameter, wherein the coolant output from the valve flows into the nozzle and a coolant spray stream is formed as a coolant passes through the nozzle;
a composition container configured to accommodate a composition;
a composition guide fluidically connected to the composition container and providing a pathway through which a composition moves, wherein an end of the composition guide disposed adjacent to the nozzle, a composition is introduced into a coolant spray stream through the composition guide when the coolant spray stream is formed by the nozzle; and
a needling structure including a body part and a plurality of needles protruding from the body part,
wherein the needling structure is disposed at a predetermined distance from the orifice of the nozzle and is disposed such that a central axis of the nozzle does not pass through the needling structure.

13. The needling and mixing spray system of claim 12, wherein a region corresponding to the nozzle and a region corresponding to the needling structure do not overlap when viewed from a front of the nozzle.

14. The needling and mixing spray system of claim 12, wherein the needling structure is disposed at a first distance from the orifice of the nozzle in a direction parallel to the central axis of the nozzle, and at a second distance from the central axis of the nozzle.

15. The needling and mixing spray system of claim 14, wherein the first distance has a value between 3 mm and 100 mm.

16. The needling and mixing spray system of claim 12, further comprising:
an additional needling structure,
wherein the additional needling structure is disposed such that the central axis of the nozzle does not pass through the additional needling structure, and
wherein the central axis of the nozzle is located between the needling structure and the additional needling structure.
